SEQUENCE LISTING (23 sequences)

SEQ ID NO: 1 - DNA, 954 bp, homo sapiens
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 2 - DNA, 5369 bp, artificial sequence
  pET-28(+) CRALBP
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - protein, 317 aa, homo sapiens
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMVDMLQDSFPARFKAIHFIHQPWYFTTTYNVVKPFLKSKLLERVFVHGDDLSGFYQEIDENILPSDFGGTLPKYDGKAVAEQLFGPQAQAENTAF

SEQ ID NO: 4 - DNA, 40 bp, artificial sequence
  primer fw_A212C
catgcagcagtgtgctagcctccggacttcagatctcagg

SEQ ID NO: 5 - DNA, 41 bp, artificial sequence
  primer rv_A212C
ggaggctagcacactgctgcatggtaaagcccttgaagttc

SEQ ID NO: 6 - DNA, 40 bp, artificial sequence
  primer fw_T250C
cttcaccacgtgctacaatgtggtcaagcccttcttgaag

SEQ ID NO: 7 - DNA, 41 bp, artificial sequence
  primer rv_T250C
ccacattgtagcacgtggtgaagtaccatggctggtggatg

SEQ ID NO: 8 - DNA, 954 bp, artificial sequence
  A212CT250C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctacaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcagtgtgctagcctccggacttcagatctcaggaagatggtggacatgctccaggattccttcccagcccggttcaaagccatccacttcatccaccagccatggtacttcaccacgtgctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 9 - protein, 317 aa, artificial sequence
  A212CT250C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQCASLRTSDLRKMVDMLQDSFPARFKAIHFIHQPWYFTTCYNVVKPFLKSKLLERVFVHGDDLSGFYQEIDENILPSDFGGTLPKYDGKAVAEQLFGPQAQAENTAF

SEQ ID NO: 10 - DNA, 954 bp, artificial sequence
  T217CV253C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggtgttcagatctcaggaagatggtggacatgctccaggattccttcccagcccggttcaaagccatccacttcatccaccagccatggtacttcaccacgacctacaattgtgtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 11 - protein, 317 aa, artificial sequence
  T217CV253C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRCSDLRKMVDMLQDSFPARFKAIHFIHQPWYFTTTYNCVKPFLKSKLLERVFVHGDDLSGFYQEIDENILPSDFGGTLPKYDGKAVAEQLFGPQAQAENTAF

SEQ ID NO: 12 - DNA, 954 bp, artificial sequence
  L220CV254C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagattgcaggaagatggtggacatgctccaggattccttcccagcccggttcaaagccatccacttcatccaccagccatggtacttcaccacgacctacaatgtgtgcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 13 - protein, 317 aa, artificial sequence
  L220CV254C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDCRKMVDMLQDSFPARFKAIHFIHQPWYFTTTYNVCKPFLKSKLLERVFVHGDDLSGFYQEIDENILPSDFGGTLPKYDGKAVAEQLFGPQAQAENTAF

SEQ ID NO: 14 - DNA, 954 bp, artificial sequence
  V224CF257C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatgtgtgacatgctccaggattccttcccagcccggttcaaagccatccacttcatccaccagccatggtacttcaccacgacctacaatgtggtcaagccctgcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 15 - protein, 317 aa, artificial sequence
  V224CF257C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMCDMLQDSFPARFKAIHFIHQPWYFTTTYNVVKPCLKSKLLERVFVHGDDLSGFYQEIDENILPSDFGGTLPKYDGKAVAEQLFGPQAQAENTAF

SEQ ID NO: 16 - DNA, 954 bp, artificial sequence
  L220CV254C - V224CF257C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagattgtaggaagatgtgtgacatgctccaggattccttcccagcccggttcaaagccatccacttcatccaccagccatggtacttcaccacgacctacaatgtgtgcaagccctgcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 17 - protein, 317 aa, artificial sequence
  L220CV254C - V224CF257C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDCRKMCDMLQDSFPARFKAIHFIHQPWYFTTTYNVCKPCLKSKLLERVFVHGDDLSGFYQEIDENILPSDFGGTLPKYDGKAVAEQLFGPQAQAENTAF

SEQ ID NO: 18 - DNA, 954 bp, artificial sequence
  A212CT250C - V224CF257C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctacaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcagtgtgctagcctccggacttcagatctcaggaagatgtgtgacatgctccaggattccttcccagcccggttcaaagccatccacttcatccaccagccatggtacttcaccacgtgctacaatgtggtcaagccctgcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 19 - protein, 317 aa, artificial sequence
  A212CT250C - V224CF257C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQCASLRTSDLRKMCDMLQDSFPARFKAIHFIHQPWYFTTCYNVVKPCLKSKLLERVFVHGDDLSGFYQEIDENILPSDFGGTLPKYDGKAVAEQLFGPQAQAENTAF

SEQ ID NO: 20 - DNA, 954 bp, artificial sequence
  A212C-T250C - T217CV253C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctacaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcagtgtgctagcctccggtgttcagatctcaggaagatggtggacatgctccaggattccttcccagcccggttcaaagccatccacttcatccaccagccatggtacttcaccacgtgctacaattgtgtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 21 - protein, 317 aa, artificial sequence
  A212C-T250C - T217CV253C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQCASLRCSDLRKMVDMLQDSFPARFKAIHFIHQPWYFTTCYNCVKPFLKSKLLERVFVHGDDLSGFYQEIDENILPSDFGGTLPKYDGKAVAEQLFGPQAQAENTAF

SEQ ID NO: 22 - DNA, 954 bp, artificial sequence
  212-220-224C250-254-257C
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctacaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcagtgtgctagcctccggacttcagattgcaggaagatgtgtgacatgctccaggattccttcccagcccggttcaaagccatccacttcatccaccagccatggtacttcaccacgtgctacaatgtgtgcaagccctgcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 23 - protein, 317 aa, artificial sequence
  212-220-224C250-254-257C
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQCASLRTSDCRKMCDMLQDSFPARFKAIHFIHQPWYFTTCYNVCKPCLKSKLLERVFVHGDDLSGFYQEIDENILPSDFGGTLPKYDGKAVAEQLFGPQAQAENTAF

(19) United States
(12) Patent Application Publication
(10) Pub. No.: US 2024/0228559 A1
(43) Pub. Date: Jul. 11, 2024

(54) REDOX SENSITIVE CRALBP MUTANT PROTEINS

(71) Applicant: UNIVERSITÄT BERN, Bern (CH)

(72) Inventor: Achim STOCKER, Oberbottigen (CH)

(21) Appl. No.: 18/561,525

(22) PCT Filed: May 18, 2022
(86) PCT No.: PCT/EP2022/063473

§ 371 Date: Nov. 16, 2023

(30) Foreign Application Priority Data
May 19, 2021 (EP) 21174665.6

(51) Int. Cl. C07K 14/47; C07K 1/02

(52) U.S. Cl. CPC C07K 14/47; C07K 1/02

(57) ABSTRACT

The present invention relates to compositions comprising complexes of CRALBP mutant proteins and CRALBP cognate ligands, wherein said CRALBP mutant proteins are muteins of wild-type CRALBP proteins comprising one or more pairs of amino acid mutations by cysteines, which pairs of cysteines are able of forming disulfide bonds, as well as to said complexes and said CRALBP mutant proteins. Moreover, the present invention further relates to methods of preparing said compositions and complexes.

The present invention relates to compositions comprising complexes of CRALBP mutant proteins and CRALBP cognate ligands, wherein said CRALBP mutant proteins are muteins of wild-type CRALBP proteins comprising one or more pairs of amino acid mutations by cysteines, which pairs of cysteines are capable of forming disulfide bonds, as well as to said complexes and said CRALBP mutant proteins. Moreover, the present invention further relates to methods of preparing said compositions and complexes.

### RELATED ART

Cellular retinal binding protein (CRALBP) was discovered in 1977 by Futterman et al. in the soluble fraction of bovine retina and shown to exhibit maximal binding for 11-cis-retinal when presented to cis-trans isomers of retinal (Futterman S., et al., 1977, J. Biol. Chem. 252(10):3267-3271). In a study by Crouch et al., the formation of rhodopsin, isorhodopsin and isorhodopsin II was demonstrated by combining the visual pigment apo-protein opsin with 11-cis-, 9-cis- and 9,13-di-cis-retinal respectively (Crouch R., et al., 1975, PNAS. 72(4):1538-1542). Together, these findings paved the way towards the assumption that CRALBP may be involved in the regeneration of bleached rhodopsin in the eye (Crouch R., et al., 1975, PNAS. 72(4): 1538-1542; Futterman S. & Saari J. C., 1977, Invest. Ophthalmol. Vis. Sci. 16(8):768-71; Crouch R., et al., 1978, Invest. Ophthalmol. Vis. Sci. 17(10):1024-9). CRALBP was isolated from bovine retina and was identified as a high affinity binder with nanomolar affinities for 9-cis-retinal (K_{d}=53 nM), 11-cis-retinal (K_{d}=20 nM) and 11-cis-retinol (K_{d}=60 nM) however does not bind to either 13-cis or all-trans-retinal. Moreover, the ratio of the first order rate constants for photoisomerization of CRALBP and rhodopsin, both in complex with 11-cis-retinal, together with the known values for the extinction coefficients of both protein complexes were used to estimate an expected 4% photoisomerization in CRALBP compared to rhodopsin (Saari J. C., et al., 1982, J. Biol. Chem., 257(22):13329-13333; Saari J C., & Bredberg D. L., 1987, J. Biol. Chem. 262(16): 7618-22). In 1988, the cloning and sequencing of the cDNA's of bovine and human CRALBP were described (Crabb J. W., et al., 1988, J. Biol. Chem. 263(35):18688-18692) and subsequently mutations of the gene encoding CRALBP were identified and associated with several retinal diseases including retinitis pigmentosa, Newfoundland rod-cone dystrophy, fundus albipunctatus, and Bothnia retinal dystrophy (Maw M. A., et al., 1997, Nat. Genet. 17:198-200; Golovleva I., et al., 2003, J. Biol. Chem. 278(14): 12397-12402; Burstedt, M. S. et al., 2003 Vision Res. 43:2559-2571; Saari, J. C.; Crabb, J. 2005, W. Exp. Eye Res. 81:245-246). X-ray structural elucidation of wild-type CRALBP and of its Bothnia dystrophy associated mutant R234W, both complexed with 11-cis-retinal, was reported by He et al. (He, X., et al., 2009, PNAS, 106(44): 18545-18550), while the X-ray structure of wild-type CRALBP complexed with 9-cis-retinal was solved by Bolze et al. further demonstrating its ability to internally convert bound 9-cis-retinal into 9,13-di-cis-retinal (Bolze, C. S., et al., 2014, JACS, 136(1):137-146). CRALBP's involvement in the retinoid visual cycle, i.e. the eye's biochemical regeneration pathway from all-trans-retinal to 11-cis-retinal, was reported (Kiser P D et al., 2014, Chem. Rev. 114:194-232; Kiser, P. D. et al., 2015 Nat. Chem. Biol. 11:409-415). In this pathway CRALBP stimulates the isomerase activity of RPE65, facilitates binding of 11-cis-retinol into the RDH5 dehydrogenase and chaperones translocation of 11-cis-retinal from RDH5 through the cytoplasm out of the cell. Studies on how CRALBP supports the mammalian retinal visual cycle and cone vision using RLBP1^{-/-} knock out mice were reported identifying Müller cell CRALBP as a key component of the retinal visual cycle and demonstrate that this pathway is important for maintaining normal cone-driven vision and accelerating cone dark adaptation (Xue, Y., et al., 2015, J. Clin. Inv., 125(2):727-738).

### SUMMARY OF THE INVENTION

The present invention describes novel engineered CRALBP mutant proteins having one or more pairs of amino acid mutations by cysteines as compared to the underlying wild-type CRALBP proteins, which pairs of cysteines are capable of forming disulfide bonds. Such redox-sensitive CRALBP mutant proteins allow production of complexes of said novel CRALBP mutant proteins with their cognate ligands, in particular cis-retinoids and preferably cis-retinals, exhibiting significantly increased photoresistance to daylight in their oxidized states. These complexes have further shown to possess similar biophysical properties compared to wild-type CRALBP under reducing conditions. Therefore, the use of such compositions and complexes, preferably the oxidized complexes, as storage devices may open new ways for an efficient and safe delivery of, preferably, cis-retinoids to retinal photoreceptors.

In a first embodiment and example, the present invention describes the dual replacement of alanine 212 and threonine 250 within the native structure of human CRALBP by cysteine residues (A212C:T250C), and its binding to 9-cis-retinal and 11-cis-retinal. The A212C modification of CRALBP maps to helix 10 of the protein's core, while the T250C modification to helix 12 within the protein's mobile gate moiety spanning residues E243 to E254.

Native CRALBP binds both 11-cis-retinal and 9-cis-retinal ligands with high affinity in the low nanomolar range. Upon ligand binding, CRALBP's mobile gate moiety adopts its ‘closed’ conformational state with helix 12 and helix 10 being within van der Waals distance of one another. In this conformation, the side-chain sulphur atoms of the mutated residues C212 and C250 oppose each other at 2.4 Å distance within the interface formed by the two helices (see FIG. 1).

We have found that both distance and orientation of the two opposing cysteine side-chains lend themselves to the formation of a covalent disulfide bond. As a direct consequence, both the exposure to atmospheric oxygen and the addition of suitable oxidizing agents induce the formation of a covalent disulfide linkage via the loss of two electrons (one for each sulphur atom). The resulting immobilization of the mobile gate in A212C:T250C provides enhanced photoprotection for the bound ligand (11-cis-retinal or 9-cis-retinal) under persisting oxidizing conditions, e.g. upon entry into extracellular compartments. In relation to the activity profile of native CRALBP, stabilization of the photosensitive cis-retinoids not only impairs the formation, but also the premature release of trans-retinal from the CRALBP binding-pocket.

Thus, the A212C:T250C point mutation foments the reversible formation of an intramolecular disulfide bond. Consistent with this observation, photo-isomerization assays reveal a significant increase in photoprotection for cis-retinal ligands when bound to the A212C:T250C mutant in its oxidized state whereas, in its reduced state, the A212C:T250C mutant possesses photoprotective properties similar to those observed with native CRALBP (see FIG. 9).

Analogous hereto, further di-cysteine mutants of CRALBP have been identified by computational methods through systematic in silico introduction of cysteine residues and analysis of the resulting energy minimized structural models containing disulfide bonds.

Thus, in a first aspect, the present invention provides a composition comprising, preferably consisting of, a complex, wherein said complex comprises (a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond; and (b) a cognate ligand of CRALBP, wherein preferably said cognate ligand is a cis-retinoid.

In a further aspect, the present invention provides a complex, wherein said complex comprises (a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond; and (b) a cognate ligand of CRALBP, wherein preferably said cognate ligand is a cis-retinoid.

In another aspect, the present invention provides a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond, wherein preferably said CRALBP mutant protein has an amino acid sequence selected from group consisting of SEQ ID NO:9, SEQ ID NO:11, SEQ ID NO: 13, SEQ ID NO: 15, SEQ ID NO:17, SEQ ID NO:19, SEQ ID NO:21 and SEQ ID NO:23, and wherein further preferably said CRALBP mutant protein has an amino acid sequence selected from SEQ ID NO:9 and SEQ ID NO:21, and wherein again further preferably said CRALBP mutant protein has the amino acid sequence of SEQ ID NO:9.

In a further aspect, the present invention provides a nucleic acid sequence encoding for a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond.

In again another aspect, the present invention provides a method of preparing a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond;
(b) a cognate ligand of CRALBP, wherein preferably said cognate ligand is a cis-retinoid; wherein said composition is preferably defined as described herein; and wherein said method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   v. separating said composition comprising said complex from said solution III;
   vi. optionally purifying said composition comprising said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In again a further aspect, the present invention provides for a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond;
(b) a cognate ligand of CRALBP, wherein preferably said cognate ligand is a cis-retinoid; wherein said composition is preferably defined as described herein; and wherein said composition is obtained by a method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   v. separating said composition comprising said complex from said solution III;
   vi. optionally purifying said composition comprising said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

Further aspects and embodiments of the present invention will be become apparent as this description continues.

### DESCRIPTION OF FIGURES

FIG. 1: Close up view of the mobile gate region of CRALBP (closed conformation) with the position and orientation of the di-cysteine A212C:T250C mutation highlighted as sticks. Mutations were introduced using Coot and side chain rotamers arranged using the program's rotamer library. Distances of 1.9-3.1 Å between the terminal sulfhydryl groups of C212 and C250 are conducive with disulfide bond (2.05 Å) formation under oxidizing conditions. 3-D overlay with the X-ray structural model (loiz.pdb) of the mobile gate moiety of the apo-form of alpha-TTP illustrates the extent of the conformational change between the closed and the open state.

FIG. 2: Close up view of the in silico model of the mobile gate region of the four mono di-cysteine mutants of CRALBP with the position of the formed di-sulfide bonds highlighted as sticks.

FIG. 3: Close up view of the in silico model of the mobile gate region of the three double and the triple di-cysteine mutant of CRALBP with the position of the formed di-sulfide bonds highlighted as sticks.

FIG. 4: Superposition of all in silico di-cysteine mutant models showing four possible di-sulfide bonds across the mobile gate.

FIG. 5: Chromatogram of preparative GFC on a Superdex® 200 26/60 after loading CRALBP di-cysteine mutant A212C:T250C with 9-cis-retinal.

FIG. 6: UV-Vis absorption spectra of monomeric wild type CRALBP and the A212C:T250C mutant in complex with 9-cis retinal. Spectra are normalized to the maximum absorption, of the protein at approximately 280 nm. While this value corresponds to the protein concentration, the absorption maximum for 9-cis retinal bound to CRALBP is at 400 nm. The fully saturated CRALBP in complex with 9-cis retinal is reported to feature an ideal spectral ratio of ε²⁸⁰/ε⁴⁰⁰=2.2. The measured ratios for wild-type CRALBP:9-cis retinal and for the A212C:T250C:9-cis retinal mutant are 2.31 and 2.21, respectively.

FIG. 7: Overlay of analytical GFC traces at 280 nm of monomeric, HMW, and SHMW fractions of CRALBP di-cysteine mutant A212C:T250C after pooling and concentrating the corresponding fraction from the preparative GFC.

FIG. 8: Photostability half-lives (t_{1/2}) of both redox states of the di-cysteine CRALBP mutant in complex with cis-retinal, respectively. The inventive di-cysteine mutant A212C:T250C within CRALBP represents a redox switch that locks the visual pigment increasing its photostability 20-fold at 1000 lux. Half-lives were calculated using the standard formula for first-order reactions (t_{1/2})=Ln(2)/R).

FIG. 9: Photoisomerization of the oxidized mutant A212C:T250C as oxidized using 5 mM oxidized glutathione. The preformed complex was oxidized overnight at 4° C. using 5 mM oxidized glutathione. From these data, reaction velocities for the different molecules have been determined for comparison purposes. The values are as follows: k (wild-type)=6.324*10⁻⁵s⁻¹, k (ox. A212C:T250C: 11-cis-retinal)=0.644*10⁻⁵s⁻¹, k (red. A212C:T250C: 11-cis-retinal)=5.988*10⁻⁵s⁻¹. No photoisomerization was detectable in the case of oxidized A212C:T250C:9-cis-retinal.

FIG. 10: Image of Native PAGE featuring individual fractions from the preparative GFC of CRALBP di-cysteine mutant A212C:T250C after loading with 9-cis-retinal. Description of lanes: M, Marker (Invitrogen™ NativeMark™); P1 and P1.2 SHMW fractions (5-8); P2 HMW Fractions (12-13); P4 monomeric fraction (26).

FIG. 11: Bar chart showing the average size, including error (+SD), of different complex fractions of CRALBP di-cysteine mutant A212C:T250C after loading with 9-cis-retinal such as SHWM, HMW, dimeric, and monomeric complex fractions, respectively.

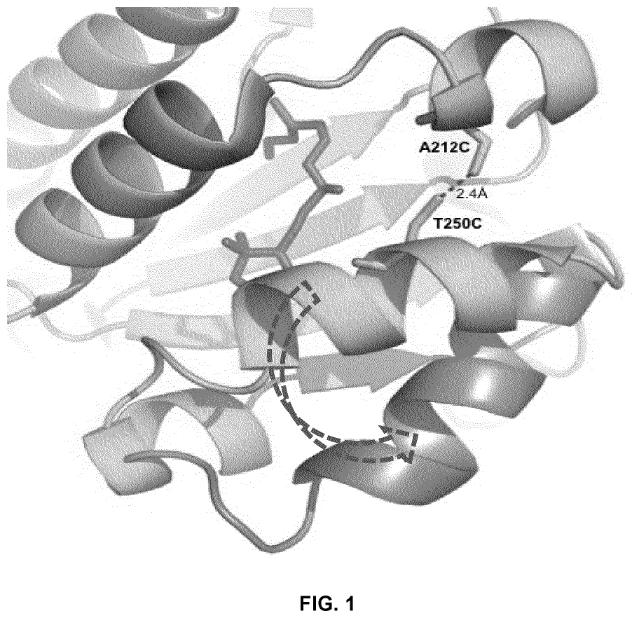

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise. As used herein, the terms “about” when referring to any numerical value are intended to mean a value of ±10% of the stated value. In a preferred embodiment, said “about” when referring to any numerical value are intended to mean a value of ±5% of the stated value. In another preferred embodiment, said “about” when referring to any numerical value are intended to mean a value of ±3% of the stated value.

Wild-type CRALBP protein: As used herein, the term “wild-type protein” refers to a cellular retinal binding protein (CRALBP) as occurred in nature for an animal, preferably a mammal, and further preferably for a human. Thus, preferably wild-type CRALBP protein refers to human wild-type CRALBP protein of SEQ ID NO:3.

Mutein: As used herein, the term “mutein” refers to a protein or polypeptide differing by one or more amino acids from a given reference (e.g. natural, wild-type, etc.) protein or polypeptide, wherein such difference is caused by addition, substitution or deletion of at least one amino acid or a combination thereof. Preferred embodiments comprise mutations derived from substitution of at least one amino acid, preferably derived from conservative substitution of at least one amino acid. Conservative substitutions include isosteric substitutions, substitutions where the charged, polar, aromatic, aliphatic or hydrophobic nature of the amino acid is maintained. For example, substitution of a cysteine residue with a serine residue is a conservative substitution. In preferred embodiments of the present invention, the term “mutein” refers to a mutein of a wild-type CRALBP protein having a sequence identity of at least 90% with said wild-type CRALBP protein, or to a mutein of a wild-type CRALBP protein differing by at most 30, typically and preferably by at most 20 or 10 amino acids from said wild-type CRALBP protein. In a further preferred embodiments of the present invention, the term “mutein” refers to a mutein of a wild-type CRALBP protein, preferably of SEQ ID NO:3, having a sequence identity of at least 90%, 91%, 92%, 93%, 94, 95%, 96% with said wild-type CRALBP protein, preferably of SEQ ID NO:3, or to a mutein of a wild-type CRALBP protein, preferably of SEQ ID NO:3, differing by at most 30, typically and preferably by at most 20, or 10, 9, 8, 7, 6 amino acids from said wild-type CRALBP protein, preferably of SEQ ID NO:3.

Position corresponding to amino acid residues . . . : The position on an amino acid sequence, which is corresponding to given residues of another amino acid sequence can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment. By way of example, in a preferred embodiment of the present invention, one cysteine of each pair of amino acid mutations by cysteines is a mutation of an amino acid within the amino acid residues corresponding to amino acids 204-229 of SEQ ID NO:3, wherein the other mutated amino acid by cysteine of said pair is a mutation of an amino acid within the amino acid residues corresponding to amino acids 244-261 of SEQ ID NO:3. Taking into account that SEQ ID NO:3 refers to human wild-type CRALBP, the corresponding specific animal or mammal CRALBP positions are therefore corresponding to said human wild-type CRALBP.

The terms “able of forming a disulfide bond” and “capable of forming a disulfide bond”, as interchangeably used herein and referring to pairs of cysteines being amino acid mutations as compared to the wild-type CRALBP protein, typically and preferably refer to the ability and capability, respectively, of the mutated cysteines to form disulfide bonds as typically and preferably determined in a manner as described in Example 3.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term amino acid exchange refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence. In case of an amino acid exchange within the present invention and referring to typically and preferably non-functional amino acid substitutions, conservative amino acid substitutions are preferred. Conservative amino acid substitutions, as understood by a skilled person in the art, include, and typically and preferably consist of isosteric substitutions, substitutions where the charged, polar, aromatic, aliphatic or hydrophobic nature of the amino acid is maintained. Typical conservative substitutions are substitutions between amino acids within one of the following groups: Gly, Ala; Val, Ile, Leu; Asp, Glu; Asn, Gln; Ser, Thr, Cys; Lys, Arg; and Phe and Tyr.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. The term “polypeptide” as used herein should also refer, typically and preferably to a polypeptide as defined before and encompassing modifications such as post-translational modifications, including but not limited to glycosylations. In a preferred embodiment, said term “polypeptide” as used herein should refer to a polypeptide as defined before and not encompassing modifications such as post-translational modifications such as glycosylations. In particular, for said biologically active peptides, said modifications such as said glycosylations can occur even in vivo thereafter, for example, by bacteria.

A cognate ligand of CRALBP: The term “a cognate ligand of CRALBP”, as used herein, refers to a molecule that binds the binding pocket of wild-type CRALBP, preferably wild-type human CRALBP of SEQ ID NO:3, with at least nanomolar affinity of typically and preferably 1-200 nM, further preferably of 1-100 nM, further typically and preferably with an affinity of 6-80 nM (wherein said affinity is typically and preferably determined as described in Golovleva I., et al., 2003, J. Biol. Chem. 278(14)), and which molecule is typically and preferably functionally associated with the CRALBP protein. Functionally associated: The term “functionally associated”, as used herein, refers to the physiological function of the CRALBP.

A cis-retinoid: The term “a cis-retinoid”, as used herein, refers to a natural or synthetic molecule, typically and preferably a Vitamin A derivative, comprising a cyclohexen or a phenyl moiety, typically and preferably substituted by one or more methyl and/or methoxy substituents, which cyclohexen or phenyl moiety is (further) substituted by a C6-C14 linear or branched alkenyl group having at its terminal carbon atom (terminal carbon atom in relation to its attachment to the cyclohexen or phenyl moiety) an alcohol, aldehyde, carboxy or an ester functionality, wherein said linear or branched alkenyl group have at least one carbon-carbon double bond, typically and preferably one or more, most preferably four carbon-carbon double bonds, wherein at least one of said carbon-carbon double bond is in the cis configuration. Said cis-retinoid includes retinol, retinaldehyde, and tretinoin, isotretinoin, alitretinoin, etretinate, acitretin as well as its esters, of which retinol and retinaldehyde are preferred. Cis-retinoids have been described (Mukherjee S et al., 2006, Clin Interv Aging. 1(4):327-48; Kiser P D et al., 2014, Chem. Rev. 114:194-232).

A “complex”: The term “a complex”, as used herein, in particular if referred to a complex comprising a CRALBP mutant protein and a cognate ligand of CRALBP, refers to (i) a 1:1 monomeric complex of said CRALBP mutant protein and said cognate ligand of CRALBP, wherein said cognate ligand of CRALBP binds said CRALBP mutant protein, typically and preferably with at least nanomolar affinity of 1-200 nM, further preferably of 1-100 nM (wherein said affinity is typically and preferably determined as described in Golovleva I., et al., 2003, J. Biol. Chem. 278(14)); and/or (ii) to a dimeric and/or oligomeric complex of said CRALBP mutant protein and said cognate ligand of CRALBP, wherein it is believed, without being bound by this theory, that typically and preferably said 1:1 monomeric complexes dimerizes and/or oligomerizes forming said dimeric and/or oligomeric complexes of said CRALBP mutant protein and said cognate ligand of CRALBP. Typically and preferably, the number of said cognate ligands within said dimeric and/or oligomeric complexes of said CRALBP mutant protein and said cognate ligand of CRALBP are equal to the number of said CRALBP mutant proteins. Further, without being bound, it is believed that the interaction between said CRALBP mutant protein and said cognate ligand of CRALBP corresponds to the stereospecific interaction as identified earlier, in particular for wild-type CRALBP protein and said cognate ligand of CRALBP such as 9-cis-retinal and 11-cis-retinal, representing reversible non-covalent interactions typical and preferably including hydrogen bonds, hydrophobic forces, van der Waals forces, electrostatic interactions or the like (He, X., et al., 2009, PNAS, 106(44): 18545-18550); Bolze, C. S., et al., 2014, JACS, 136(1):137-146).

Animal: The term “animal”, as used herein, may be an animal (e.g., a non human animal), a vertebrate animal, a mammal, a rodent (e.g., a guinea pig, a hamster), a canine (e.g., a dog), a feline (e.g., a cat), a porcine (e.g., a pig), an equine (e.g., a horse), a primate, or a human. In a preferred embodiment said animal is a mammal. In another preferred embodiment said animal is a human or a non-human mammal (such as, e.g., a dog, a cat, a horse). In a very preferred embodiment said animal is a human.

In a first aspect, the present invention provides a composition comprising, preferably consisting of, a complex, wherein said complex comprises (a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond; and (b) a cognate ligand of CRALBP, wherein preferably said cognate ligand is a cis-retinoid.

In a further aspect, the present invention provides a complex, wherein said complex comprises (a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond; and (b) a cognate ligand of CRALBP, wherein preferably said cognate ligand is a cis-retinoid.

In another aspect, the present invention provides a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond, wherein preferably said CRALBP mutant protein has an amino acid sequence selected from group consisting of SEQ ID NO:9, SEQ ID NO:11, SEQ ID NO: 13, SEQ ID NO: 15, SEQ ID NO:17, SEQ ID NO: 19, SEQ ID NO:21 and SEQ ID NO:23, and wherein further preferably said CRALBP mutant protein has an amino acid sequence selected from SEQ ID NO:9 and SEQ ID NO:21, and wherein again further preferably said CRALBP mutant protein has the amino acid sequence of SEQ ID NO:9.

In a further aspect, the present invention provides a nucleic acid sequence encoding for a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond.

In again another aspect, the present invention provides a method of preparing a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said composition is preferably defined as described herein; and wherein said method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   v. separating said composition comprising said complex from said solution III; optionally purifying said composition comprising said complex; and vi.
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In again a further aspect, the present invention provides for a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said composition is preferably defined as described herein; and wherein said composition is obtained by a method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   V. separating said composition comprising said complex from said solution III;
   vi. optionally purifying said composition comprising said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a method of preparing a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said composition is preferably defined as described herein in preferred embodiments; and wherein said method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5;
   ii. providing said cognate ligand of CRALBP in an aqueous solution II, wherein the concentration of said cognate ligand of CRALBP in said solution I is 5 μM to 500 mM; and wherein said solution II comprises a detergent;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar);
   iv. removing said detergent from said solution III, wherein removing said detergent from said solution III allows said allows said CRALBP mutant protein and said cognate ligand of CRALBP to assemble into a complex;
   v. separating composition comprising said complex from said solution III;
   vi. optionally purifying said composition comprising said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a method of preparing a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said complex is preferably defined as described herein in preferred embodiments; and wherein said method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   v. separating said complex from said solution III;
   vi. optionally purifying said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a method of preparing a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said complex is preferably defined as described herein in preferred embodiments; and wherein said method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5;
   ii. providing said cognate ligand of CRALBP in an aqueous solution II, wherein the concentration of said cognate ligand of CRALBP in said solution I is 5 μM to 500 mM; and wherein said solution II comprises a detergent;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar);
   iv. removing said detergent from said solution III, wherein removing said detergent from said solution III allows said allows said CRALBP mutant protein and said cognate ligand of CRALBP to assemble into a complex;
   v. separating said complex from said solution III;
   vi. optionally purifying said complex, and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said composition is preferably defined as described herein in preferred embodiments; and wherein said composition is obtained by a method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5;
   ii. providing said cognate ligand of CRALBP in an aqueous solution II, wherein the concentration of said cognate ligand of CRALBP in said solution I is 5 μM to 500 mM; and wherein said solution II comprises a detergent;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar);
   iv. removing said detergent from said solution III, wherein removing said detergent from said solution III allows said allows said CRALBP mutant protein and said cognate ligand of CRALBP to assemble into a complex;
   v. separating composition comprising said complex from said solution III;
   vi. optionally purifying said composition comprising said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid; wherein said complex is preferably defined as described herein in preferred embodiments; and wherein said complex is obtained by a method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
   ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
   iv. allowing said CRALBP mutant protein and said cognate ligand of CRALBP to assemble into a complex;
   v. separating said complex from said solution III;
   vi. optionally purifying said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In a further aspect, the present invention provides a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is capable of forming a disulfide bond;
(b) a cognate ligand of CRALBP, wherein preferably said cognate ligand is a cis-retinoid; wherein said complex is preferably defined as described herein in preferred embodiments; and wherein said complex is obtained by a method comprises the steps of
   i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5;
   ii. providing said cognate ligand of CRALBP in an aqueous solution II, wherein the concentration of said cognate ligand of CRALBP in said solution I is 5 μM to 500 mM; and wherein said solution II comprises a detergent;
   iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar);
   iv. removing said detergent from said solution III, wherein removing said detergent from said solution III allows said allows said CRALBP mutant protein and said cognate ligand of CRALBP to assemble into a complex;
   v. separating said complex from said solution III;
   vi. optionally purifying said complex; and
   vii. optionally treating said composition comprising said complex with an oxidizing agent, wherein preferably said oxidizing agent is ambient air or 1-1000 mM oxidized glutathione, further preferably wherein said oxidizing agent is 1-100 mM oxidized glutathione.

In again a further aspect, the present invention provides for a pharmaceutical composition comprising (a) the composition of the present invention; and (b) a pharmaceutically acceptable carrier.

In again a further aspect, the present invention provides for a pharmaceutical composition comprising (a) the complex of the present invention; and (b) a pharmaceutically acceptable carrier.

In a preferred embodiment, said cognate ligand, preferably said cis-retinoid, is selected from (i) 9-cis-retinal; (ii) 9-cis-retinol; (iii) 11-cis-retinal; (iv) 11-cis-retinol; (v) 11, 13-di-cis-retinal; (vi) 11, 13-di-cis-retinol; (vii) 9, 13-di-cis-retinal; and (viii) 9, 13-di-cis-retinol. In a further preferred embodiment, said cognate ligand, preferably said cis-retinoid is selected from (i) 9-cis-retinal; (ii) 9-cis-retinol; (iii) 11-cis-retinal; (iv) 11-cis-retinol; (v) 9,13-di-cis-retinal; and (vi) 9,13-di-cis-retinol. In a further preferred embodiment, said cognate ligand, preferably said cis-retinoid is selected from (i) 9-cis-retinal; (ii) 11-cis-retinal; and (iii) 9,13-di-cis-retinal; and (vi) 9,13-di-cis-retinol. In a further preferred embodiment, said cognate ligand, preferably said cis-retinoid is selected from (i) 9-cis-retinal and (ii) 11-cis-retinal.

In a further preferred embodiment, said cognate ligand, preferably said cis-retinoid is 9-cis-retinal. In a further preferred embodiment, said cognate ligand, preferably said cis-retinoid is 9-cis-retinol. In a further preferred embodiment, said cognate ligand, preferably said cis-retinoid is 11-cis-retinal. In a further preferred embodiment, said cognate ligand, preferably said cis-retinoid is 11-cis-retinol. In a further preferred embodiment, said cognate ligand, preferably said cis-retinoid is 11, 13-di-cis-retinal. In a further preferred embodiment, said cognate ligand, preferably said cis-retinoid is 11, 13-di-cis-retinol. In a further preferred embodiment, said cognate ligand, preferably said cis-retinoid is 9, 13-di-cis-retinal. In a further preferred embodiment, said cognate ligand, preferably said cis-retinoid is 9, 13-di-cis-retinol.

In a further preferred embodiment said mutein of a wild-type CRALBP protein has a sequence identity of at least 90% with said wild-type CRALBP protein, preferably of at least 95%, further preferably of at least 96%, again further preferably at least 97%, again further preferably at least 98%, and again more preferably of at least 99%. In a further preferred embodiment said mutein of a wild-type CRALBP protein has a sequence identity of at least 95% with said wild-type CRALBP protein. In a further preferred embodiment said mutein of a wild-type CRALBP protein has a sequence identity of at least 96% with said wild-type CRALBP protein. In a further preferred embodiment said mutein of a wild-type CRALBP protein has a sequence identity of at least 97% with said wild-type CRALBP protein. In a further preferred embodiment said mutein of a wild-type CRALBP protein has a sequence identity of at least 98% with said wild-type CRALBP protein. In a further preferred embodiment said mutein of a wild-type CRALBP protein has a sequence identity of at least 99% with said wild-type CRALBP protein.

In a further preferred embodiment said mutein of a wild-type CRALBP protein differs by at most 30 amino acids from said wild-type CRALBP protein In a further preferred embodiment said mutein of a wild-type CRALBP protein differs by at most 20 amino acids from said wild-type CRALBP protein. In again another preferred embodiment said mutein of a wild-type CRALBP protein differs by at most 10 amino acids from said wild-type CRALBP protein. In a further preferred embodiment said mutein of a wild-type CRALBP protein differs by 10, 9, 8, 7, 6, 5, 4, 3, 2, 1 amino acids from said wild-type CRALBP protein. In a further preferred embodiment said mutein of a wild-type CRALBP protein differs by at most 8 amino acids from said wild-type CRALBP protein. In a further preferred embodiment said mutein of a wild-type CRALBP protein differs by at most 6, 4 or 2 amino acids from said wild-type CRALBP protein.

In a further preferred embodiment, one cysteine of each pair of amino acid mutations by cysteines is a mutation of an amino acid within the amino acid residues corresponding to amino acids 204-229 of SEQ ID NO:3, wherein the other mutated amino acid by cysteine of said pair is a mutation of an amino acid within the amino acid residues corresponding to amino acids 244-261 of SEQ ID NO:3.

In a further preferred embodiment, said mutein comprises one, two, three or four pairs of amino acid mutations by cysteines as compared to said wild-type CRALBP protein, and wherein preferably said mutein comprises one or two pairs of amino acid mutations by cysteines as compared to said wild-type CRALBP protein.

In a further preferred embodiment, said mutein comprises one pair of amino acid mutations by cysteines as compared to said wild-type CRALBP protein. In another preferred embodiment, said mutein comprises two pairs of amino acid mutations by cysteines as compared to said wild-type CRALBP protein. In a further preferred embodiment, said mutein comprises three pairs of amino acid mutations by cysteines as compared to said wild-type CRALBP protein.

In a further preferred embodiment, said pair of amino acid mutations by cysteines as compared to said wild-type CRALBP protein is selected from
(1) a mutation of an amino acid corresponding to amino acid 212 of SEQ ID NO:3 and a mutation of an amino acid corresponding to amino acid 250 of SEQ ID NO:3;
(2) a mutation of an amino acid corresponding to amino acid 217 of SEQ ID NO:3 and a mutation of an amino acid corresponding to amino acid 253 of SEQ ID NO:3;
(3) a mutation of an amino acid corresponding to amino acid 220 of SEQ ID NO:3 and a mutation of an amino acid corresponding to amino acid 254 of SEQ ID NO:3;
(4) a mutation of an amino acid corresponding to amino acid 224 of SEQ ID NO:3 and a mutation of an amino acid corresponding to amino acid 257 of SEQ ID NO:3.

In a further preferred embodiment, said pair of amino acid mutations by cysteines as compared to said wild-type CRALBP protein is a mutation of an amino acid corresponding to amino acid 212 of SEQ ID NO:3 and a mutation of an amino acid corresponding to amino acid 250 of SEQ ID NO:3. In a further preferred embodiment, said pair of amino acid mutations by cysteines as compared to said wild-type CRALBP protein is a mutation of an amino acid corresponding to amino acid 217 of SEQ ID NO:3 and a mutation of an amino acid corresponding to amino acid 253 of SEQ ID NO:3. In a further preferred embodiment, said pair of amino acid mutations by cysteines as compared to said wild-type CRALBP protein is a mutation of an amino acid corresponding to amino acid 220 of SEQ ID NO:3 and a mutation of an amino acid corresponding to amino acid 254 of SEQ ID NO:3. In a further preferred embodiment, said pair of amino acid mutations by cysteines as compared to said wild-type CRALBP protein is a mutation of an amino acid corresponding to amino acid 224 of SEQ ID NO:3 and a mutation of an amino acid corresponding to amino acid 257 of SEQ ID NO:3.

In a further preferred embodiment, said mutein comprises one or two or three pairs of amino acid mutations by cysteines as compared to said wild-type CRALBP protein, and wherein said one pair of amino acid mutations by cysteines as compared to said wild-type CRALBP protein is selected from
(i) a mutation of an amino acid corresponding to amino acid 212 of SEQ ID NO:3 and a mutation of an amino acid corresponding to amino acid 250 of SEQ ID NO:3;
(ii) a mutation of an amino acid corresponding to amino acid 217 of SEQ ID NO:3 and a mutation of an amino acid corresponding to amino acid 253 of SEQ ID NO:3; and
(iii) a mutation of an amino acid corresponding to amino acid 220 of SEQ ID NO:3 and a mutation of an amino acid corresponding to amino acid 254 of SEQ ID NO:3;
   and wherein said two pairs of amino acid mutations by cysteines as compared to said wild-type CRALBP protein are selected from
(a) a first pair of amino acid mutations by cysteines and a second pair of amino acid mutations by cysteines, wherein said first pair of amino acid mutations by cysteines is a mutation of an amino acid corresponding to amino acid 220 of SEQ ID NO:3 and a mutation of an amino acid corresponding to amino acid 254 of SEQ ID NO:3, and said second pair of amino acid mutations by cysteines is a mutation of an amino acid corresponding to amino acid 224 of SEQ ID NO:3 and a mutation of an amino acid corresponding to amino acid 257 of SEQ ID NO:3;
(b) a first pair of amino acid mutations by cysteines and a second pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, and said second pair of amino acid mutations by cysteines is a mutation of an amino acid corresponding to amino acid 224 of SEQ ID NO:3 and a mutation of an amino acid corresponding to amino acid 257 of SEQ ID NO:3; and
(c) a first pair of amino acid mutations by cysteines and a second pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, and said second pair of amino acid mutations by cysteines is a mutation of an amino acid corresponding to amino acid 217 of SEQ ID NO:3 and a mutation of an amino acid corresponding to amino acid 253 of SEQ ID NO:3;
   and wherein said three pairs of amino acid mutations by cysteines as compared to said wild-type CRALBP protein is
(x) a first pair of amino acid mutations by cysteines, a second pair of amino acid mutations by cysteines, and a third pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, said second pair of amino acid mutations by cysteines is a mutation of an amino acid corresponding to amino acid 220 of SEQ ID NO:3 and a mutation of an amino acid corresponding to amino acid 254 of SEQ ID NO:3, and said third pair of amino acid mutations by cysteines is a mutation of an amino acid corresponding to amino acid 224 of SEQ ID NO:3 and a mutation of an amino acid corresponding to amino acid 257 of SEQ ID NO:3.

In a further preferred embodiment, said mutein comprises one pair of amino acid mutations by cysteines as compared to said wild-type CRALBP protein, and wherein said one pair of amino acid mutations by cysteines as compared to said wild-type CRALBP protein is a mutation of an amino acid corresponding to amino acid 212 of SEQ ID NO:3 and a mutation of an amino acid corresponding to amino acid 250 of SEQ ID NO:3. In a further preferred embodiment, said mutein comprises one pair of amino acid mutations by cysteines as compared to said wild-type CRALBP protein, and wherein said one pair of amino acid mutations by cysteines as compared to said wild-type CRALBP protein is a mutation of an amino acid corresponding to amino acid 217 of SEQ ID NO:3 and a mutation of an amino acid corresponding to amino acid 253 of SEQ ID NO:3. In a further preferred embodiment, said mutein comprises one pair of amino acid mutations by cysteines as compared to said wild-type CRALBP protein, and wherein said one pair of amino acid mutations by cysteines as compared to said wild-type CRALBP protein is a mutation of an amino acid corresponding to amino acid 220 of SEQ ID NO:3 and a mutation of an amino acid corresponding to amino acid 254 of SEQ ID NO:3.

In a further preferred embodiment, said mutein comprises two pairs of amino acid mutations by cysteines as compared to said wild-type CRALBP protein, and wherein said two pairs of amino acid mutations by cysteines as compared to said wild-type CRALBP protein are a first pair of amino acid mutations by cysteines and a second pair of amino acid mutations by cysteines, wherein said first pair of amino acid mutations by cysteines is a mutation of an amino acid corresponding to amino acid 220 of SEQ ID NO:3 and a mutation of an amino acid corresponding to amino acid 254 of SEQ ID NO:3, and said second pair of amino acid mutations by cysteines is a mutation of an amino acid corresponding to amino acid 224 of SEQ ID NO:3 and a mutation of an amino acid corresponding to amino acid 257 of SEQ ID NO:3.

In a further preferred embodiment, said mutein comprises two pairs of amino acid mutations by cysteines as compared to said wild-type CRALBP protein, and wherein said two pairs of amino acid mutations by cysteines as compared to said wild-type CRALBP protein are a first pair of amino acid mutations by cysteines and a second pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, and said second pair of amino acid mutations by cysteines is a mutation of an amino acid corresponding to amino acid 224 of SEQ ID NO:3 and a mutation of an amino acid corresponding to amino acid 257 of SEQ ID NO:3.

In a further preferred embodiment, said mutein comprises two pairs of amino acid mutations by cysteines as compared to said wild-type CRALBP protein, and wherein said two pairs of amino acid mutations by cysteines as compared to said wild-type CRALBP protein are a first pair of amino acid mutations by cysteines and a second pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, and said second pair of amino acid mutations by cysteines is a mutation of an amino acid corresponding to amino acid 217 of SEQ ID NO:3 and a mutation of an amino acid corresponding to amino acid 253 of SEQ ID NO:3.

In a further preferred embodiment, said mutein comprises three pairs of amino acid mutations by cysteines as compared to said wild-type CRALBP protein, and wherein said three pairs of amino acid mutations by cysteines as compared to said wild-type CRALBP protein are a first pair of amino acid mutations by cysteines, a second pair of amino acid mutations by cysteines, and a third pair of amino acid mutations by cysteines, wherein said first pair of amino acid mutations by cysteines is a mutation of an amino acid corresponding to amino acid 212 of SEQ ID NO:3 and a mutation of an amino acid corresponding to amino acid 250 of SEQ ID NO:3, said second pair of amino acid mutations by cysteines is a mutation of an amino acid corresponding to amino acid 220 of SEQ ID NO:3 and a mutation of an amino acid corresponding to amino acid 254 of SEQ ID NO:3, and said third pair of amino acid mutations by cysteines is a mutation of an amino acid corresponding to amino acid 224 of SEQ ID NO:3 and a mutation of an amino acid corresponding to amino acid 257 of SEQ ID NO:3.

In a further preferred embodiment, said complex is a monomeric complex of said CRALBP mutant protein and said cognate ligand.

In a further preferred embodiment, said complex is a dimeric complex of said CRALBP mutant protein and said cognate ligand.

In a further preferred embodiment, said complex is an oligomeric complex of said CRALBP mutant protein and said cognate ligand. In a further preferred embodiment, said complex is an homo oligomeric complex of said CRALBP mutant protein and said cognate ligand.

In a further preferred embodiment, said complex is an oligomeric complex of said CRALBP mutant protein and said cognate ligand, wherein said oligomeric complex has a molecular weight of at least 240 kDa, preferably of at least 300 kDa, and wherein preferably said oligomeric complex has a molecular weight of at most 660 kDa, further preferably wherein said oligomeric complex has a molecular weight of at most 600 kDa.

In a further preferred embodiment, said complex is an oligomeric complex of said CRALBP mutant protein and said cognate ligand, wherein said oligomeric complex has a molecular weight of at least 720 kDa, preferably of at least 800 kDa, and wherein preferably said oligomeric complex has a molecular weight of at most 2500 kDa, further preferably wherein said oligomeric complex has a molecular weight of at most 2000 kDa.

In a further preferred embodiment, said complex is an oligomeric complex of said CRALBP mutant protein and said cognate ligand, wherein said oligomeric complex has a molecular weight of at least 720 kDa, preferably of at least 800 kDa, and wherein preferably said oligomeric complex has a molecular weight of at most 2500 kDa, further preferably wherein said oligomeric complex has a molecular weight of at most 2000 kDa.

In a further preferred embodiment, said complex is an oligomeric complex of said CRALBP mutant protein and said cognate ligand, wherein the number of said cognate ligands and said CRALBP mutant proteins within said oligomeric complex are equal or higher than 6 and equal or lower than 18, preferably equal or higher than 8 and and equal or lower than 16, and wherein further preferably said number of said cognate ligands the number of said CRALBP mutant proteins are equal.

In a further preferred embodiment, said complex is an oligomeric complex of said CRALBP mutant protein and said cognate ligand, wherein the number of said cognate ligands and said CRALBP mutant proteins within said oligomeric complex are equal or higher than 20 and equal or lower than 75, preferably equal or higher than 22 and equal or lower than 60, and wherein further preferably said number of said cognate ligands the number of said CRALBP mutant proteins are equal. In a further preferred embodiment, said complex is an oligomeric complex of said CRALBP mutant protein and said cognate ligand, wherein the number of said cognate ligands and said CRALBP mutant proteins within said oligomeric complex are equal or higher than 24 and equal or lower than 75, preferably equal or higher than 26 and equal or lower than 60, and wherein further preferably said number of said cognate ligands the number of said CRALBP mutant proteins are equal.

In a further preferred embodiment, said complex is an oligomeric complex of said CRALBP mutant protein and said cognate ligand, wherein said oligomeric complex has a average diameter of about 8 to 20 nm, wherein said average diameter is determined by Dynamic Light Scattering (DLS), and wherein preferably said oligomeric complex has a average diameter of about 10 to 18 nm, wherein said average diameter is determined by Dynamic Light Scattering (DLS).

In a further preferred embodiment, said complex is an oligomeric complex of said CRALBP mutant protein and said cognate ligand, wherein said oligomeric complex has a average diameter of about 24 to 33 nm, wherein said average diameter is determined by Dynamic Light Scattering (DLS), and wherein preferably said oligomeric complex has a average diameter of about 25 to 32 nm, wherein said average diameter is determined by Dynamic Light Scattering (DLS).

In a further preferred embodiment, said composition comprises monomeric complexes and homo oligomeric complexes of said CRALBP mutant protein and said cognate ligand. In a further preferred embodiment, said complex comprises monomeric complexes and homo oligomeric complexes of said CRALBP mutant protein and said cognate ligand.

In a preferred embodiment, at least one of said pair of cysteines forms a disulfide bond. In another preferred embodiment, each of said pair of cysteines forms a disulfide bond.

In a preferred embodiment, said wild-type CRALBP protein is the human CRALBP protein of SEQ ID NO:3.

In a preferred embodiment, said wild-type CRALBP protein is the human CRALBP protein of SEQ ID NO:3, and said pair of amino acid mutations by cysteines as compared to said human wild-type CRALBP protein is selected from
(1) a mutation of amino acid 212 of SEQ ID NO:3 and a mutation of amino acid 250 of SEQ ID NO:3;
(2) a mutation of amino acid 217 of SEQ ID NO:3 and a mutation of amino acid 253 of SEQ ID NO:3;
(3) a mutation of amino acid 220 of SEQ ID NO:3 and a mutation of amino acid 254 of SEQ ID NO:3;
(4) a mutation of amino acid 224 of SEQ ID NO:3 and a mutation of amino acid 257 of SEQ ID NO:3;
   and wherein preferably, said mutein of said human wild-type CRALBP protein has a sequence identity of at least 90% with said human wild-type CRALBP protein, preferably of at least 95%, further preferably of at least 96%, again further preferably at least 97%, again further preferably at least 98%, and again more preferably of at least 99%.

In a further preferred embodiment, said wild-type CRALBP protein is the human CRALBP protein of SEQ ID NO:3, and said mutein of said human wild-type CRALBP protein has a sequence identity of at least 95% with said human wild-type CRALBP protein. In a further preferred embodiment, said wild-type CRALBP protein is the human CRALBP protein of SEQ ID NO:3, and said mutein of said human wild-type CRALBP protein has a sequence identity of at least 96% with said human wild-type CRALBP protein. In a further preferred embodiment, said wild-type CRALBP protein is the human CRALBP protein of SEQ ID NO:3, and said mutein of said human wild-type CRALBP protein has a sequence identity of at least 97% with said human wild-type CRALBP protein. In a further preferred embodiment, said wild-type CRALBP protein is the human CRALBP protein of SEQ ID NO:3, and said mutein of said human wild-type CRALBP protein has a sequence identity of at least 98% with said human wild-type CRALBP protein. In a further preferred embodiment, said wild-type CRALBP protein is the human CRALBP protein of SEQ ID NO:3, and said mutein of said human wild-type CRALBP protein has a sequence identity of at least 99% with said human wild-type CRALBP protein.

In a further preferred embodiment, said wild-type CRALBP protein is the human CRALBP protein of SEQ ID NO:3, and said mutein of said human wild-type CRALBP protein differs by at most 30 amino acids from said human wild-type CRALBP protein. In a further preferred embodiment, said wild-type CRALBP protein is the human CRALBP protein of SEQ ID NO:3, and said mutein of said human wild-type CRALBP protein differs by at most 20 amino acids from said human wild-type CRALBP protein. In a further preferred embodiment, said wild-type CRALBP protein is the human CRALBP protein of SEQ ID NO:3, and said mutein of said human wild-type CRALBP protein differs by at most 10 amino acids from said human wild-type CRALBP protein. In a further preferred embodiment, said wild-type CRALBP protein is the human CRALBP protein of SEQ ID NO:3, and said mutein of said human wild-type CRALBP protein differs by 10, 9, 8, 7, 6, 5, 4, 3, 2, 1 amino acids from said human wild-type CRALBP protein. In a further preferred embodiment, said wild-type CRALBP protein is the human CRALBP protein of SEQ ID NO:3, and said mutein of said human wild-type CRALBP protein differs by at most 8 amino acids from said human wild-type CRALBP protein. In a further preferred embodiment, said wild-type CRALBP protein is the human CRALBP protein of SEQ ID NO:3, and said mutein of said human wild-type CRALBP protein differs by at most 6, 4 or 2 amino acids from said human wild-type CRALBP protein.

In a further preferred embodiment, said wild-type CRALBP protein is the human CRALBP protein of SEQ ID NO:3, said pair of amino acid mutations by cysteines as compared to said human wild-type CRALBP protein is a mutation of amino acid 212 of SEQ ID NO:3 and a mutation of amino acid 250 of SEQ ID NO:3. In a further preferred embodiment, said wild-type CRALBP protein is the human CRALBP protein of SEQ ID NO:3, and said pair of amino acid mutations by cysteines as compared to said human wild-type CRALBP protein is a mutation of amino acid 217 of SEQ ID NO:3 and a mutation of amino acid 253 of SEQ ID NO:3. In a further preferred embodiment, said wild-type CRALBP protein is the human CRALBP protein of SEQ ID NO:3, and said pair of amino acid mutations by cysteines as compared to said human wild-type CRALBP protein is a mutation of amino acid 220 of SEQ ID NO:3 and a mutation of amino acid 254 of SEQ ID NO:3. In a further preferred embodiment, said wild-type CRALBP protein is the human CRALBP protein of SEQ ID NO:3, and said pair of amino acid mutations by cysteines as compared to said human wild-type CRALBP protein is a mutation of amino acid 224 of SEQ ID NO:3 and a mutation of amino acid 257 of SEQ ID NO:3.

In a further preferred embodiment, said wild-type CRALBP protein is the human CRALBP protein of SEQ ID NO:3, and said mutein comprises one or two or three pairs of amino acid mutations by cysteines as compared to said human wild-type CRALBP protein, and wherein said one pair of amino acid mutations by cysteines as compared to said human wild-type CRALBP protein is selected from
(i) a mutation of amino acid 212 of SEQ ID NO:3 and a mutation of amino acid 250 of SEQ ID NO:3;
(ii) a mutation of amino acid 217 of SEQ ID NO:3 and a mutation of amino acid 253 of SEQ ID NO:3; and
(iii) a mutation of amino acid 220 of SEQ ID NO:3 and a mutation of amino acid 254 of SEQ ID NO:3;
   and wherein said two pairs of amino acid mutations by cysteines as compared to said human wild-type CRALBP protein are selected from
(a) a first pair of amino acid mutations by cysteines and a second pair of amino acid mutations by cysteines, wherein said first pair of amino acid mutations by cysteines is a mutation of amino acid 220 of SEQ ID NO:3 and a mutation of amino acid 254 of SEQ ID NO:3, and said second pair of amino acid mutations by cysteines is a mutation of amino acid 224 of SEQ ID NO:3 and a mutation of amino acid 257 of SEQ ID NO:3;
(b) a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 224 of SEQ ID NO:3 and a mutation of amino acid 257 of SEQ ID NO:3; and
(c) a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 217 of SEQ ID NO:3 and a mutation of amino acid 253 of SEQ ID NO:3;
   and wherein said three pairs of amino acid mutations by cysteines as compared to said human wild-type CRALBP protein is
(x) a first pair of amino acid mutations by cysteines, a second pair of amino acid mutations by cysteines, and a third pair of amino acid mutations by cysteines, wherein said first pair of amino acid mutations by cysteines is a mutation of amino acid 212 of SEQ ID NO:3 and a mutation of amino acid 250 of SEQ ID NO:3, said second pair of amino acid mutations by cysteines is a mutation of amino acid 220 of SEQ ID NO:3 and a mutation of amino acid 254 of SEQ ID NO:3, and said third pair of amino acid mutations by cysteines is a mutation of amino acid 224 of SEQ ID NO:3 and a mutation of amino acid 257 of SEQ ID NO:3.

In a further preferred embodiment, said wild-type CRALBP protein is the human CRALBP protein of SEQ ID NO:3, and said mutein comprises one pair of amino acid mutations by cysteines as compared to said human wild-type CRALBP protein, and wherein said one pair of amino acid mutations by cysteines as compared to said human wild-type CRALBP protein is a mutation of amino acid 212 of SEQ ID NO:3 and a mutation of amino acid 250 of SEQ ID NO:3. In a further preferred embodiment, said wild-type CRALBP protein is the human CRALBP protein of SEQ ID NO:3, and said mutein comprises one pair of amino acid mutations by cysteines as compared to said human wild-type CRALBP protein, and wherein said one pair of amino acid mutations by cysteines as compared to said human wild-type CRALBP protein is a mutation of amino acid 217 of SEQ ID NO:3 and a mutation of amino acid 253 of SEQ ID NO:3. In a further preferred embodiment, said wild-type CRALBP protein is the human CRALBP protein of SEQ ID NO:3, and said mutein comprises one pair of amino acid mutations by cysteines as compared to said human wild-type CRALBP protein, and wherein said one pair of amino acid mutations by cysteines as compared to said human wild-type CRALBP protein is a mutation of amino acid 220 of SEQ ID NO:3 and a mutation of amino acid 254 of SEQ ID NO:3.

In a further preferred embodiment, said wild-type CRALBP protein is the human CRALBP protein of SEQ ID NO:3, and said mutein comprises two pairs of amino acid mutations by cysteines as compared to said human wild-type CRALBP protein, and wherein said two pairs of amino acid mutations by cysteines as compared to said human wild-type CRALBP protein are a first pair of amino acid mutations by cysteines and a second pair of amino acid mutations by cysteines, wherein said first pair of amino acid mutations by cysteines is a mutation of amino acid 220 of SEQ ID NO:3 and a mutation of amino acid 254 of SEQ ID NO:3, and said second pair of amino acid mutations by cysteines is a mutation of amino acid 224 of SEQ ID NO:3 and a mutation of amino acid 257 of SEQ ID NO:3.

In a further preferred embodiment, said wild-type CRALBP protein is the human CRALBP protein of SEQ ID NO:3, and said mutein comprises two pairs of amino acid mutations by cysteines as compared to said human wild-type CRALBP protein, and wherein said two pairs of amino acid mutations by cysteines as compared to said human wild-type CRALBP protein are a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 224 of SEQ ID NO:3 and a mutation of amino acid 257 of SEQ ID NO:3.

In a further preferred embodiment, said wild-type CRALBP protein is the human CRALBP protein of SEQ ID NO:3, and said mutein comprises two pairs of amino acid mutations by cysteines as compared to said human wild-type CRALBP protein, and wherein said two pairs of amino acid mutations by cysteines as compared to said human wild-type CRALBP protein are a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 217 of SEQ ID NO:3 and a mutation of amino acid 253 of SEQ ID NO:3.

In a further preferred embodiment, said wild-type CRALBP protein is the human CRALBP protein of SEQ ID NO:3, and said mutein comprises three pairs of amino acid mutations by cysteines as compared to said human wild-type CRALBP protein, and wherein said three pairs of amino acid mutations by cysteines as compared to said human wild-type CRALBP protein are a first pair of amino acid mutations by cysteines, a second pair of amino acid mutations by cysteines, and a third pair of amino acid mutations by cysteines, wherein said first pair of amino acid mutations by cysteines is a mutation of amino acid 212 of SEQ ID NO:3 and a mutation of amino acid 250 of SEQ ID NO:3, said second pair of amino acid mutations by cysteines is a mutation of amino acid 220 of SEQ ID NO:3 and a mutation of amino acid 254 of SEQ ID NO:3, and said third pair of amino acid mutations by cysteines is a mutation of amino acid 224 of SEQ ID NO:3 and a mutation of amino acid 257 of SEQ ID NO:3.

In a further preferred embodiment, said CRALBP mutant protein has an amino acid sequence selected from group consisting of SEQ ID NO:9, SEQ ID NO:11, SEQ ID NO:13, SEQ ID NO:15, SEQ ID NO: 17, SEQ ID NO: 19, SEQ ID NO:21 and SEQ ID NO:23, and wherein preferably said CRALBP mutant protein has an amino acid sequence selected from SEQ ID NO:9 and SEQ ID NO:21, and wherein further preferably said CRALBP mutant protein has the amino acid sequence of SEQ ID NO:9. In another preferred embodiment of the aforementioned embodiments, each of said pair of cysteines forms a disulfide bond, and thus, within said inventive complexes, said CRALBP mutant protein is completely in the oxidized form.

In a further preferred embodiment, said CRALBP mutant protein has the amino acid sequence of SEQ ID NO:9. In a further preferred embodiment, said CRALBP mutant protein has the amino acid sequence of SEQ ID NO:11. In a further preferred embodiment, said CRALBP mutant protein has the amino acid sequence of SEQ ID NO: 13. In a further preferred embodiment, said CRALBP mutant protein has the amino acid sequence of SEQ ID NO: 15. In a further preferred embodiment, said CRALBP mutant protein has the amino acid sequence of SEQ ID NO: 17. In a further preferred embodiment, said CRALBP mutant protein has the amino acid sequence of SEQ ID NO: 19. In a further preferred embodiment, said CRALBP mutant protein has the amino acid sequence of SEQ ID NO:21. In a further preferred embodiment, said CRALBP mutant protein has the amino acid sequence of SEQ ID NO:23. In another preferred embodiment of the aforementioned embodiments, each of said pair of cysteines forms a disulfide bond, and thus, within said inventive complexes, said CRALBP mutant protein is completely in the oxidized form. In a further preferred embodiment, said CRALBP mutant protein comprises, preferably consists of, the amino acid sequence of SEQ ID NO:9, and wherein said pair of cysteines of amino acid 212 and 250 of SEQ ID NO:9 forms a disulfide bond.

In a further preferred embodiment, said CRALBP mutant protein consists of an amino acid sequence selected from group consisting of SEQ ID NO:9, SEQ ID NO: 11, SEQ ID NO:13, SEQ ID NO:15, SEQ ID NO:17, SEQ ID NO:19, SEQ ID NO:21 and SEQ ID NO:23, and wherein preferably said CRALBP mutant protein consists of an amino acid sequence selected from SEQ ID NO:9 and SEQ ID NO:21, and wherein further preferably said CRALBP mutant protein consists of the amino acid sequence of SEQ ID NO:9. In another preferred embodiment of the aforementioned embodiments, each of said pair of cysteines forms a disulfide bond. Thus, within said inventive complexes, said CRALBP mutant protein is completely in the oxidized form.

In a further preferred embodiment, said CRALBP mutant protein consists of the amino acid sequence of SEQ ID NO:9. In a further preferred embodiment, said CRALBP mutant protein consists of the amino acid sequence of SEQ ID NO:11. In a further preferred embodiment, said CRALBP mutant protein consists of the amino acid sequence of SEQ ID NO:13. In a further preferred embodiment, said CRALBP mutant protein consists of the amino acid sequence of SEQ ID NO:15. In a further preferred embodiment, said CRALBP mutant protein consists of the amino acid sequence of SEQ ID NO:17. In a further preferred embodiment, said CRALBP mutant protein consists of the amino acid sequence of SEQ ID NO: 19. In a further preferred embodiment, said CRALBP mutant protein consists of the amino acid sequence of SEQ ID NO:21. In a further preferred embodiment, said CRALBP mutant protein consists of the amino acid sequence of SEQ ID NO:23. In another preferred embodiment of the aforementioned embodiments, each of said pair of cysteines forms a disulfide bond. Thus, within said inventive complexes, said CRALBP mutant protein is completely in the oxidized form. In a further preferred embodiment, said CRALBP mutant protein consists of the amino acid sequence of SEQ ID NO:9, and wherein said pair of cysteines of amino acid 212 and 250 of SEQ ID NO:9 forms a disulfide bond.

In a further preferred embodiment, said composition comprising, preferably consisting of, a complex, wherein said complex comprises (a) a CRALBP mutant protein, wherein said CRALBP mutant protein comprises, preferably consists of, the amino acid sequence of SEQ ID NO:9; and (b) a cognate ligand of CRALBP, wherein said cognate ligand is a cis-retinoid, wherein preferably said cis-retinoid is selected from (i) 9-cis-retinal; (ii) 9-cis-retinol; (iii) 11-cis-retinal; (iv) 11-cis-retinol; (v) 9,13-di-cis-retinal; and (vi) 9,13-di-cis-retinol, further preferably wherein said cis-retinoid is selected from (i) 9-cis-retinal; (ii) 11-cis-retinal; and (iii) 9,13-di-cis-retinal; and (vi) 9,13-di-cis-retinol, and again further preferably wherein said cis-retinoid is selected from (i) 9-cis-retinal and (ii) 11-cis-retinal; and wherein preferably said pair of cysteines of amino acid 212 and 250 of SEQ ID NO:9 forms a disulfide bond.

In a further preferred embodiment, said composition comprising, preferably consisting of, a complex, wherein said complex comprises (a) a CRALBP mutant protein, wherein said CRALBP mutant protein consists of the amino acid sequence of SEQ ID NO:9; and (b) a cognate ligand of CRALBP, wherein said cognate ligand is a cis-retinoid selected from (i) 9-cis-retinal and (ii) 11-cis-retinal; and wherein preferably said pair of cysteines of amino acid 212 and 250 of SEQ ID NO:9 forms a disulfide bond, and wherein preferably said complex is an oligomeric complex of said CRALBP mutant protein and said cognate ligand, wherein said oligomeric complex has a molecular weight of at least 720 kDa, preferably of at least 800 kDa, and wherein preferably said oligomeric complex has a molecular weight of at most 2500 kDa, further preferably wherein said oligomeric complex has a molecular weight of at most 2000 kDa.

In a further preferred embodiment, said composition comprising, preferably consisting of, a complex, wherein said complex comprises (a) a CRALBP mutant protein, wherein said CRALBP mutant protein consists of the amino acid sequence of SEQ ID NO:9; and (b) a cognate ligand of CRALBP, wherein said cognate ligand is a cis-retinoid selected from (i) 9-cis-retinal and (ii) 11-cis-retinal; and wherein preferably said pair of cysteines of amino acid 212 and 250 of SEQ ID NO:9 forms a disulfide bond, and wherein preferably said complex is an oligomeric complex of said CRALBP mutant protein and said cognate ligand, wherein said oligomeric complex has a molecular weight of at least 800 kDa, and wherein said oligomeric complex has a molecular weight of at most 2000 kDa.

In a further preferred embodiment, said composition comprising, preferably consisting of, a complex, wherein said complex comprises (a) a CRALBP mutant protein, wherein said CRALBP mutant protein consists of the amino acid sequence of SEQ ID NO:9; and (b) a cognate ligand of CRALBP, wherein said cognate ligand is a cis-retinoid selected from (i) 9-cis-retinal and (ii) 11-cis-retinal; and wherein said pair of cysteines of amino acid 212 and 250 of SEQ ID NO:9 forms a disulfide bond, and wherein said complex is an oligomeric complex of said CRALBP mutant protein and said cognate ligand, wherein said oligomeric complex has a molecular weight of at least 720 kDa, preferably of at least 800 kDa, and wherein preferably said oligomeric complex has a molecular weight of at most 2500 kDa, further preferably wherein said oligomeric complex has a molecular weight of at most 2000 kDa.

In a further preferred embodiment, said composition comprising, preferably consisting of, a complex, wherein said complex comprises (a) a CRALBP mutant protein, wherein said CRALBP mutant protein consists of the amino acid sequence of SEQ ID NO:9; and (b) a cognate ligand of CRALBP, wherein said cognate ligand is a cis-retinoid selected from (i) 9-cis-retinal and (ii) 11-cis-retinal; and wherein said pair of cysteines of amino acid 212 and 250 of SEQ ID NO:9 forms a disulfide bond, and wherein preferably said complex is an oligomeric complex of said CRALBP mutant protein and said cognate ligand, wherein said oligomeric complex has a molecular weight of at least 800 kDa, and wherein said oligomeric complex has a molecular weight of at most 2000 kDa.

In a further preferred embodiment, the concentration of said CRALBP mutant protein in said solution I is 0.1 mM to 1 mM, and further preferably the concentration of said CRALBP mutant protein in said solution I is 0.25 mM to 0.75 mM. In a further preferred embodiment, the pH of said solution I is 7 to 8.5, further preferably 7.5 to 8.5.

In a further preferred embodiment, said solution I comprises a salt, wherein the concentration of said salt is 10 mM to 500 mM. Further preferred, the concentration of said salt is 30 mM to 350 mM, and further preferably the concentration of said salt is 100 mM to 250 mM. In a further preferred embodiment, said salt is selected from a mono-, di-, tri- or tetravalent inorganic or organic salt, and wherein further preferably said salt is selected from a di-, tri- or tetravalent inorganic or organic salt, and wherein again further preferably said salt comprises a divalent, tri, or tetravalent anion selected from HPO₄²⁻, SO₄²⁻, tartrate, malonate, D-myo-inositol 1,4,5-triphosphate and D-myo-inositol 1,3,4,5-tetrakis(phosphate) potassium salt.

In a further preferred embodiment, said concentration of said cognate ligand of CRALBP in said solution I is 30 μM to 300 mM, further preferably the concentration of said cognate ligand of CRALBP in said solution I is 200 μM to 200 mM.

The solvent of said solution II is a water soluble solvent. It is within the scope of the present invention that the water soluble solvent used for solution II may comprise minor amounts, i.e. up to 10% (v/v) of water even though typically and preferably said water soluble solvent does not comprise any additional amounts of water other than the minor amount of water typically comprised in said water soluble solvent when supplied by the manufacturer. Further preferably, said water soluble solvent dose not comprise more than 8% (v/v), preferably more than 7% (v/v), further preferably more than 5% (v/v) of water.

In a preferred embodiment of the present invention, said water soluble solvent is selected from methanol, ethanol, isopropanol, propanol, butanol, dimethyl sulfoxide (DMSO), tetrahydrofuran (THF), dioxan, methylpentane diol and glycerol; further preferably said water soluble solvent is selected from ethanol, isopropanol and dimethyl sulfoxide (DMSO), again further preferably said water soluble solvent is ethanol.

For the inventive methods, wherein said solution II comprises a detergent, said detergent serves to solubilize said cognate ligand of CRALBP in said solution II. In a preferred embodiment, said detergent is selected from a non-ionic detergent or an anionic detergent. Preferably said non-ionic detergent is selected from octyl beta-D-glucoside, nonyl beta-D-glucoside, decyl beta-D-glucoside, nonyl beta-D-maltoside, decyl beta-D-maltoside, undecyl beta-D-maltoside, dodecyl beta-D-maltoside, Tween 20R, Tween 40R, Tween 80R Triton X100R, Nonidet P40, polyethylenglycol 200. Preferably said anionic detergent is selected from sodium cholate, sodium deoxycholate, sodium glycocholate, sodium deoxyglycocholate and sodium taurocholate. In a further preferred embodiment, said non-ionic detergent is selected from octyl beta-D-glucoside, nonyl beta-D-glucoside, decyl beta-D-glucoside, nonyl beta-D-maltoside, decyl beta-D-maltoside, undecyl beta-D-maltoside, dodecyl beta-D-maltoside, Tween 20R, Tween 40R, Tween 80R Triton X100R, Nonidet P40, polyethylenglycol 200. In a further preferred embodiment, said anionic detergent is selected from sodium cholate, sodium deoxycholate, sodium glycocholate, sodium deoxyglycocholate and sodium taurocholate. In a preferred embodiment, said detergent is an anionic detergent, wherein said anionic detergent is sodium cholate.

The concentration of said detergent used in said solution II can be determined by the skilled person in the art and is based on the knowledge the skilled person in the art since in order to solubilize said cognate ligand of CRALBP in said solution II the concentration is dependent on the nature of the detergent used. Typically and preferably the concentration of said detergent in said solution II is higher the critical micelle concentration (CMC) of said detergent, typically and preferably of said non-ionic detergent or said anionic detergent. Typically and preferably the critical micelle concentration (CMC) of said detergent, typically and preferably of non-ionic detergent or said anionic detergent is equal or higher than 0.5 mM, and wherein further preferably the critical micelle concentration (CMC) of said non-ionic detergent or said anionic detergent is equal or higher than 10 mM.

In a further preferred embodiment, said removing of said detergent from said solution III is performed by dialysis; and wherein preferably said removing of said detergent from said solution III by dialysis is performed across a membrane, wherein preferably said membrane comprises a molecular weight cut off of 1 to 25 kD, preferably of 5 to 20 kD, and again further preferably of 10-15 kD. The dialysis is preferably performed with a first buffer, wherein said first buffer comprises a halogenide of an alkaline metal, wherein preferably said halogenide of an alkaline metal is potassium chloride or sodium chloride, and wherein further preferably said halogenide of an alkaline metal is sodium chloride, wherein preferably the concentration of said halogenide of an alkaline metal, preferably said sodium chloride in said first buffer is 1 to 1000 mM, preferably 10 to 500 mM, more preferably 50 to 250 mM, most preferably 100-200 mM.

The dialysis is preferably performed at a temperature of 4° C. to 37° C., and wherein preferably said dialysis is performed over a period of 4 to 24h.

In a preferred embodiment of the present invention, said ratio of the concentration of said CRALBP mutant protein and the concentration of said cognate ligand of said CRALBP in said solution III is of between 3:1 to 1:3 (molar/molar), and preferably the ratio of the concentration of said CRALBP mutant protein and the concentration of said cognate ligand of said CRALBP in said solution III is of between 2:1 to 1:2 (molar/molar).

In another preferred embodiment of the present invention, said volume of said water soluble solvent in said solution III is of between 1-5% (vol/vol), and preferably the volume of said water soluble solvent in said solution III is of between 2-4% (vol/vol).

In a preferred embodiment of the present invention, said allowing said CRALBP mutant protein and said cognate ligand of said CRALBP to assemble into a complex is effected by keeping said solution III at a temperature of 4-37° C., preferably at room temperature, for a period of 1h to 24h, preferably for a period of 12 to 24h.

In a further preferred embodiment of the present invention, said separating said composition comprising said complex, or separating said complex, from said solution III is effected by size exclusion chromatography or anionic exchange chromatography, preferably by size exclusion chromatography.

In another preferred embodiment of the present invention, said purifying said composition comprising said complex, or purifying said complex, is effected by size exclusion chromatography or anionic exchange chromatography, preferably by size exclusion chromatography. In another preferred embodiment of the present invention, said purifying said composition comprising said complex, or purifying said complex, is effected by size exclusion chromatography or anionic exchange chromatography under oxidizing conditions, preferably by size exclusion chromatography under oxidizing conditions, further preferably as described in Example 8 using oxidized glutathione.

In another preferred embodiment of the present invention, said method comprises purifying said composition comprising said complex, or purifying said complex, by size exclusion chromatography or anionic exchange chromatography under oxidizing conditions, preferably by size exclusion chromatography under oxidizing conditions, further preferably as described in Example 8 using oxidized glutathione. In another preferred embodiment of the present invention, said method comprises purifying said composition comprising said complex, or purifying said complex, by size exclusion chromatography or anionic exchange chromatography under oxidizing conditions, preferably by size exclusion chromatography under oxidizing conditions, wherein said size exclusion chromatography or anionic exchange chromatography, preferably said size exclusion chromatography, is effected in presence of an oxidizing agent, wherein preferably said oxidizing agent is oxidized glutathione, and wherein preferably the concentration of said oxidizing agent, preferably of said oxidized glutathione, is between 1 mM and 7.5 mM, preferably between 3 mM and 6 mM, and further preferably about 5 mM. In a further preferred embodiment of the inventive method, said composition comprising, preferably consisting of, a complex, wherein said complex comprises (a) a CRALBP mutant protein, wherein said CRALBP mutant protein consists of the amino acid sequence of SEQ ID NO:9; and (b) a cognate ligand of CRALBP, wherein said cognate ligand is a cis-retinoid selected from (i) 9-cis-retinal and (ii) 11-cis-retinal; and wherein said pair of cysteines of amino acid 212 and 250 of SEQ ID NO:9 forms a disulfide bond, and wherein said complex is an oligomeric complex of said CRALBP mutant protein and said cognate ligand, wherein said oligomeric complex has a molecular weight of at least 720 kDa, preferably of at least 800 kDa, and wherein preferably said oligomeric complex has a molecular weight of at most 2500 kDa, further preferably wherein said oligomeric complex has a molecular weight of at most 2000 kDa, and said method comprises purifying said composition comprising said complex, or purifying said complex, by size exclusion chromatography or anionic exchange chromatography under oxidizing conditions, preferably by size exclusion chromatography under oxidizing conditions, further preferably as described in Example 8 using oxidized glutathione. In another preferred embodiment of the present invention, said method comprises purifying said composition comprising said complex, or purifying said complex, by size exclusion chromatography or anionic exchange chromatography under oxidizing conditions, preferably by size exclusion chromatography under oxidizing conditions, wherein said size exclusion chromatography or anionic exchange chromatography, preferably said size exclusion chromatography, is effected in presence of an oxidizing agent, wherein preferably said oxidizing agent is oxidized glutathione, and wherein preferably the concentration of said oxidizing agent, preferably of said oxidized glutathione, is between 1 mM and 7.5 mM, preferably between 3 mM and 6 mM, and further preferably about 5 mM.

### EXAMPLES

### Example 1

### Cloning of RLBP1 and Expression of CRALBP

Cloning of RLBP1 and expression of CRALBP was analogously effected as described in He et al. (He, X., et al., 2009, PNAS, 106(44): 18545-18550). In brief, human RLBP1 cDNA (JRAUp969D1020D, SEQ ID NO:1) was obtained from the German Center for Genomic Research GmbH. The CRALBP overexpression vector construct was obtained by cloning the RLBP1 cDNA into the NdeI and XhoI sites of the pET-28a(+) vector following the protocol of “StreamLined Restriction Digestion, Dephosphorylation and Ligation” (Promega) leading to the pET-28a(+) CRALBP overexpression plasmid (SEQ ID NO:2). Incubations were carried out at 37° C. and 750 rpm (Thermomixer Compact, Eppendorf) for 2.25h, inactivation at 74° C. for 15 min, separation via 1% agarose gel and extraction of the desired cutting fragments with the “Wizard SV Gel and PCR Clean-Up System” from Promega according to the user manual. Thermosensitive Alkaline Phosphatase was used only for the digestion of the pET-28a(+) target vector but not for the digestion of the RLBP1 cDNA (JRAUp969D1020D). BL21(DE3) strains of E. coli (Invitrogen) were transformed with the pET-28a(+) CRALBP overexpression plasmid (SEQ ID NO:2), and were cultured overnight with agitation at 37° C. in 120 mL LB medium containing 30 mg/mL kanamycin. The overnight culture was used to inoculate 6 L of LB medium (30 mg/mL kanamycin). The culture was grown at 20° ° C. to an OD600 of 0.7 and then was induced with 1 mM isopropyl-thiogalacto-pyranoside for 16 h. Cells were harvested by centrifugation at 5000 g for 45 min and were resuspended in 250 mL of ice-cold lysis buffer (20 mM imidazole; 100 mM NaCl; 20 mM Tris-HCl, pH 7.4; 1% wt/vol Triton X-100). The cells were disrupted by ultrasonication for 20 min, and the lysate comprising human wild-type CRALPB (SEQ ID NO:3) was centrifuged at 20,000 g for 35 min to remove debris.

### Example 2

### Cloning and Expression of Di-Cys Mutant CRALBP A212C:T250C

Point mutations were introduced sequentially by site-directed mutagenesis in pET-28a(+) CRALBP overexpression plasmid (SEQ ID NO:2) according to the instructions described in the QuikChange kit from Stratagene. The corresponding primer sequences, the PCR reaction mixtures and the temperature protocol used to produce the di-cys mutant CRALBP A212C:T250C are depicted in Table 1 and Table 2 respectively. Thus, in Table 1 mutated positions are identified by underlined-blocks. In the case of T250C, the point mutation directly leads to the formation of a novel restriction site. In the case of the A212C point mutation, an additional silent mutation was introduced (see underlined and cursive).

TABLE 1
Primer sequences

| Primer | | | Restriction |
|---|---|---|---|
| name | Mutation | Primer 5′-3′ | site |
| fw_A212C | A212C | catgcagcagtgtg | BmtI |
| | | ctagcctccggact | |
| | | tcagatctcagg | |
| | | (SEQ ID NO: 4) | |
| rv_A212C | A212C | ggaggctagcacac | BmtI |
| | | tgctgcatggtaaa | |
| | | gcccttgaagttc | |
| | | (SEQ ID NO: 5) | |
| fw_T250C | T250C | cttcaccacgtgct | PmlI |
| | | acaatgtggtcaag | |
| | | cccttcttgaag | |
| | | (SEQ ID NO: 6) | |
| rv_T250C | T250C | ccacattgtagcac | PmlI |
| | | gtggtgaagtacca | |
| | | tggctggtggatg | |
| | | (SEQ ID NO: 7) | |

TABLE 2
PCR reaction mixtures and conditions

| PCR mix | Volume | | PCR program | Temperature | Time | |
|---|---|---|---|---|---|---|
| Template DNA SEQ ID NO: 2 | 100 | ng | Initial denaturation | 94° C. | 3 | min |
| 10× Pfu buffer | 5 | μl | Denaturation | 94° C. | 18 × 1 | min |
| Forward primer (5 μM) | 0.2 | μl | Annealing | 55° C. | 18 × 1 | min |
| Reverse primer (5 μM) | 0.2 | μl | Elongation | 68° C. | 18 × 20 | min |
| 2 mM dNTP mix | 5 | μl | Final Elongation | 68° C. | 18 × 60 | min |
| Pfu Polymerase | 2 | μl | End Hold | 10° C. | — | |
| ad H₂O | 50 | μl | | | | |

Thus, nucleotide sequence of SEQ ID NO:2 was used as starting template in the site-directed PCR. After sequential PCR reactions the resulting nucleotide sequence of SEQ ID NO:8 encoding for the amino acid SEQ ID NO:9 of the di-cys mutant CRALBP A212C:T250C was obtained.

Thus, after PCR reaction, 2 μl of DpnI restriction enzymes were added to the reaction mixture and the sample incubated for two hours at 37ºC. Following this, 10 μl of the reaction mixture was directly transformed into competent E. coli XL10Gold cells (Stratagene). Clones obtained after transformation and plating were checked for the presence of freshly introduced restriction sites by analytical restriction digestion. Clones containing both restriction sites were used for isolating the di-cys mutant CRALBP A212C: T250C overexpression plasmid of SEQ ID NO:8. The presence of the A212C:T250C point mutations in the plasmid was verified by sequencing (Microsynth AG, Balgach).

BL21(DE3) cells transformed with and containing the di-cys mutant CRALBP A212C:T250C overexpression plasmid of SEQ ID NO:8 were cultured overnight with agitation at 37° C. in 120 mL LB medium containing 30 mg/mL kanamycin. The overnight culture was used to inoculate 6 L of LB medium (30 mg/mL kanamycin). The culture was grown at 20° C. to an OD600 of 0.7 and then was induced with 1 mM isopropyl-thiogalacto-pyranoside for 16 h. Cells were harvested by centrifugation at 5000 g for 45 min and were resuspended in 250 ml of ice-cold lysis buffer (20 mM imidazole; 100 mM NaCl; 20 mM Tris-HCl, pH 7.4; 1% wt/vol Triton X-100). The cells were disrupted by ultrasonication for 20 min, and the lysate comprising the di-cys mutant CRALBP A212C: T250C of SEQ ID NO:9 was centrifuged at 20,000 g for 35 min to remove debris.

### Example 3

### In Silico Mutagnesis of CRALBP

In silico cysteine mutagenesis was carried out in order to identify possibly stabilizing disulfide bridges in CRALBP, other than the above described di-cysteine mutant A212C: T250C (SEQ ID NO:9). For this, a truncated model was created from the X-ray structural model of wild-type CRALBP with bound 11-cis-retinal (PDB entry 3HY5). Two sequence regions of wild-type CRALBP (SEQ ID NO:3), namely amino acid residues 204-229 of SEQ ID NO:3) and amino acid residues 244-261 of SEQ ID NO:3 were defined by editing the original model. Cysteine modifications were introduced systematically into both chains using the FoldX V5 computer algorithm (Schymkowitz J., et al., 2005, Nucleic Acids Res. 1; 33:W382-8. doi: 10.1093/nar/gki387). Using the algorithm, the truncated model of wild-type CRALBP was minimized at 298K, pH 7, 0.1 M ionic strength to generate an in silico reference structure (“Reference—WT”). The FoldX V5 “BuildModel” command, which implements a probability-based rotamer library of its own, was used to create in silico eight CRALBP mutant proteins comprising one, two or three pairs of cysteines mutations containing models including the di-cys mutant A212C:T250C. Table 3 lists said identified CRALBP mutant proteins as well as their amino acid and nucleic acid sequences.

TABLE 3
Overview of di-cysteine mutants of CRALBP including
amino acid and nucleotide sequences

| | Pairs | | |
|---|---|---|---|
| | of di-cys | Nucleic Acid | Amino Acid |
| Mutants | mutations | Sequence | Sequence |
| A212C:T250C | mono di-cys | SEQ ID NO: 8 | SEQ ID NO: 9 |
| T217C:V253C | mono di-cys | SEQ ID NO: 10 | SEQ ID NO: 11 |
| L220C:V254C | mono di-cys | SEQ ID NO: 12 | SEQ ID NO: 13 |
| V224C:F257C | mono di-cys | SEQ ID NO: 14 | SEQ ID NO: 15 |
| L220C:V254C- | double | SEQ ID NO: 16 | SEQ ID NO: 17 |
| V224C:F257C | di-cys | | |
| A212C:T250C- | double | SEQ ID NO: 18 | SEQ ID NO: 19 |
| V224C:F257C | di-cys | | |
| A212C-T250C- | double | SEQ ID NO: 20 | SEQ ID NO: 21 |
| T217C:V253C | di-cys | | |
| 212-220-224C:250- | triple | SEQ ID NO: 22 | SEQ ID NO: 23 |
| 254-257C | di-cys | | |

The mutant models from FoldX were submitted to phenix.dynamic (Adams P. D., et al., 2010, Acta Crystallogr. D Biol. Crystallogr. 66(Pt 2):213-21) for simple model perturbation and flexible backbone modeling respectively, thereby de-biasing the models and simultaneously forming the disulfide bonds. Molecular dynamics calculations were performed at 300K, 200 iterations of 0.005 fs each, and the threshold maximum for bond formation set to 3.1 Å. Subsequently, the change in free energy of the interface was assessed with the EMBL PISA server17. The solvation free energy gain upon formation of the interface between the two layers of the mobile gate (residues 204-229 of SEQ ID NO:3 and residues 244-261 of SEQ ID NO:3) was calculated as AG in kcal/mol. A compilation of the calculations for the described inventive CRALBP mutants including the wild-type CRALBP (SEQ ID NO:3) as reference is shown in Table 4.

TABLE 4
Overview of in silico mutant analysis

| | | | | Distance SS- | |
|---|---|---|---|---|---|
| | Chain A | Chain B | interface | Bonds | ΔiG |
| Variant | Surface Å² | Surface Å² | area Å² | Å | kcal/mol |
| Reference wt CRALBP | 2837 | 2060 | 472.4 | NA | −11.2 |
| V224C:F257C | 2853 | 2099 | 490.8 | 2.021 | −9.7 |
| T217C:V253C | 2815 | 2085 | 487.7 | 2.052 | −12.6 |
| L220C:V254C | 2923 | 2096 | 481.0 | 2.084 | −12.8 |
| A212C:T250C | 2861 | 2120 | 524.9 | 2.042 | −15.3 |
| L220C:V254C-V224C:F257C | 2921 | 2122 | 487.1 | 2.030/2.037 | −12.6 |
| A212C:T250C-V224C:F257C | 2810 | 2109 | 531.8 | 2.043/2.049 | −13.7 |
| A212C-T250C-T217C-V253C | 2885 | 2138 | 569.0 | 2.062/2.056 | −17.4 |
| 212-220-224C:250-254-257C | 2864 | 2127 | 536.4 | 2.062/2.053/2.001 | −17.3 |

Surface Å² is the total solvent accessible surface area in square Angstroms: Interface area in A2, calculated as difference in total accessible surface areas of isolated and interfacing structures divided by two. ΔⁱG indicates the solvation free energy gain upon formation of the interface, in kcal/mol. Any ΔⁱG value that is reported to be more negative than the reference value of −11.2 kcal/mol is considered to effect a further stabilization of the corresponding mutant in comparison to wild-type CRALBP.

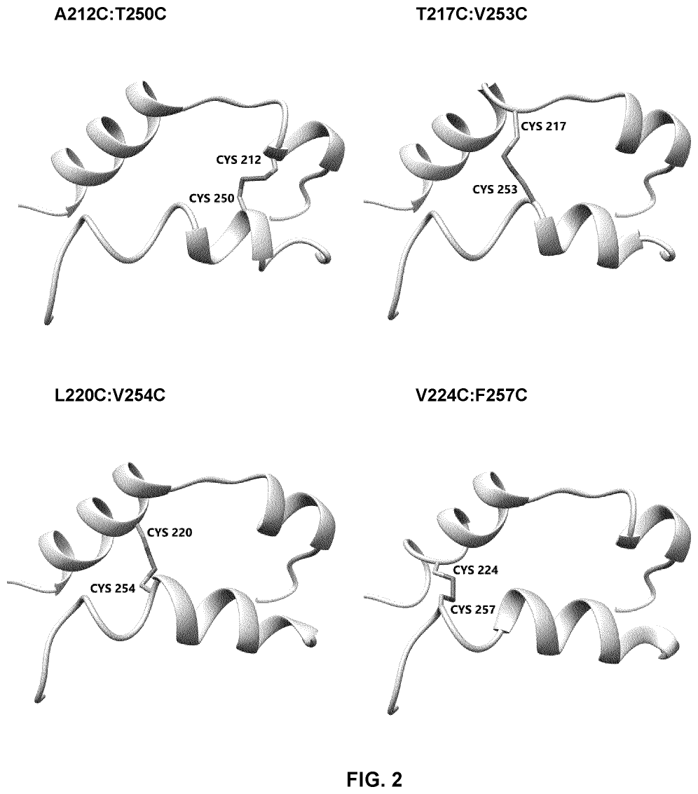

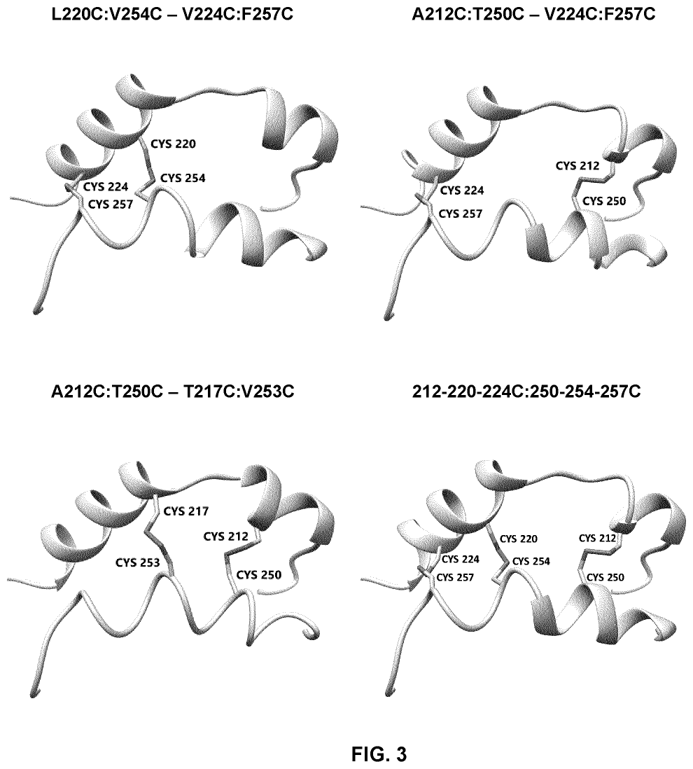

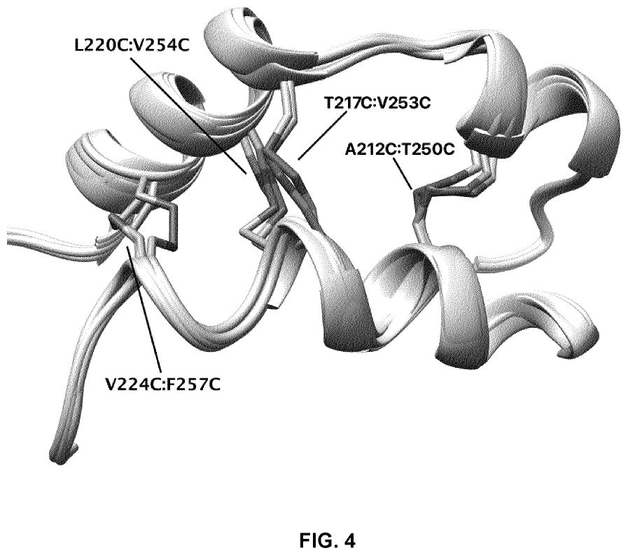

FIG. 2 shows individually the close up view of the in silico model of the mobile gate region of the four described mono di-cysteine mutants of CRALBP with the position of the formed disulfide bonds highlighted as sticks, while in FIG. 3 the close up view of the in silico model of the mobile gate region of each of the three double and the triple di-cysteine mutants of CRALBP with the position of the formed disulfide bonds highlighted as sticks are shown. The superposition of all in silico di-cysteine mutant models showing four possible described disulfide bonds across the mobile gate of CRALBP is shown in FIG. 4.

### Example 4

### Cloning and Expression of the in Silico Generated Di-Cys CRALBP Mutants

The nucleic acid sequences as well as the amino acid sequences of the further identified di-cys CRALBP mutants are described in Table 3. The cloning of the corresponding mutant RLBP 1 gene sequences and the expression of the corresponding di-cys CRALBP mutant proteins is effected analogously as described in Example 1 and 2. The purification of these further identified di-cys CRALBP mutants is effected as described in Example 5 below.

### Example 5

### Purification of Di-Cys CRALBP Mutants

The lysate comprising the di-cys mutant CRALBP A212C:T250C of SEQ ID NO:9 obtained in Example 2 was purified from the supernatant by affinity chromatography on 10 mL of Ni-NTA SUPERFLOW (Qiagen) according to the manufacturer's instructions. Briefly, the lysate was loaded on the column previously equilibrated in lysis buffer, was washed with lysis buffer, and was eluted in 35 mL of elution buffer (20 mM Tris-HCl, pH 7.4; 200 mM imidazole; 100 mM NaCl). Typical yields were 35-40 mg of pure di-cys mutant CRALBP A212C:T250C as judged by SDS/PAGE and determined using the colorimetric bicinchoninic acid assay (Pierce Chemical Company).

### Example 6

### Ligand Loading and Gel Permeation Chromatography

All procedures involving cis-retinal were performed under dim red illumination (40-W ruby bulbs) at 4° C. unless specified. Ligand loading of the affinity purified wild-type CRALBP and of the di-cysteine mutant A212C:T250C of CRALBP was carried out in elution buffer (20 mM Tris-HCl, pH 7.4; 200 mM imidazole; 100 mM NaCl) by adding aliquots of 9-cis-retinal or 11-cis-retinal stocks (40 mM) dissolved in ethanol to the protein solutions at a 1.5 molar excess and a final ethanol concentration of 2% vol/vol. The samples were incubated for 30 min at 4° C. and then centrifuged at 15,000 g for 10 min. The samples were concentrated using a Vivaspin 15R Hydrosart (Sartorius) to 30-50 mg/ml of protein with three washes in buffer (10 mM Tris-HCl, 100 mM NaCl, pH 7.5). Finally, unbound retinoid was removed from the ligand complexes by gel filtration chromatography (GFC).

### Example 7

### Gel Filtration Chromatography of Di-Cys Mutants of CRALBP

Purification of the protein cis-retinal complex of di-cysteine mutant A212C:T250C of CRALBP was performed on a Superdex® 200 26/60 column in Gel filtration chromatography (GFC) buffer (10 mM Hepes, 100 mM NaCl, pH 7.5). The total elution volume was 330 ml, and fractions were collected at 5 ml intervals. Preparative gel filtration reveals essentially the same chromatogram as for the cis-retinal complex of wild-type CRALBP.

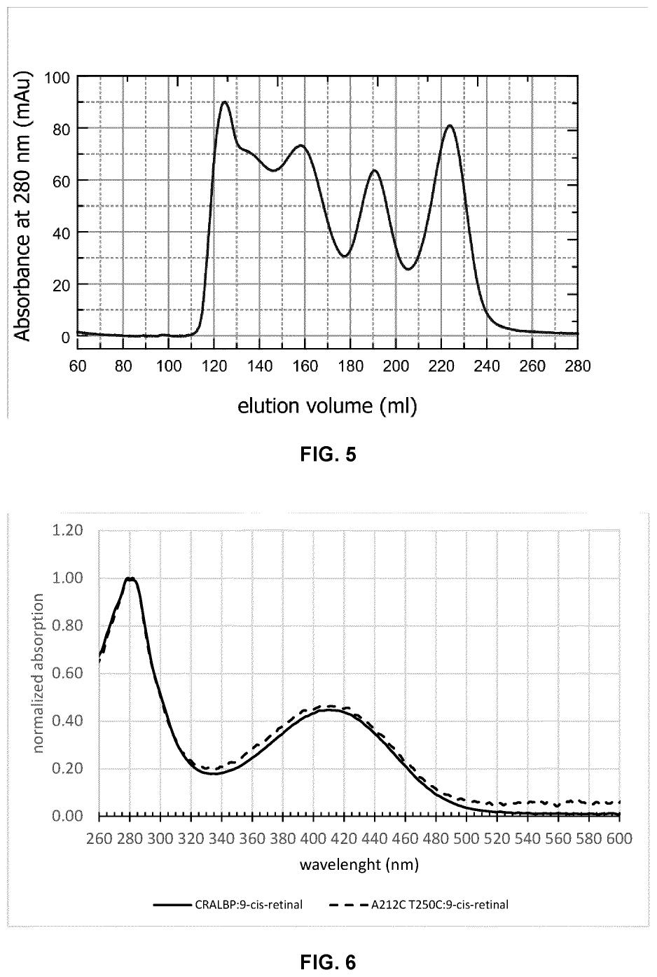

Again, four significant peaks with similar elution volumes can be observed when the apo di-cysteine mutant A212C:T250C of CRALBP are loaded with cis-retinal. FIG. 5 shows the typical UV/Vis absorption trace at 280 nm featuring said four peaks. The order of the peaks is as follows: the first peak from left to right corresponds to the super high molecular weight (SHMW) fractions, the second peak corresponds to the high molecular weight (HMW) fraction, the third peak corresponds to dimeric, and the fourth peak to monomeric fractions, respectively. UV-Vis absorption spectra of monomeric wild type CRALBP and of the A212C: T250C mutant in complex with 9-cis retinal were used to determine the ligand loading of the monomeric complexes. For this, the absorption spectra were normalized to the maximum absorption, of the protein at approximately 280 nm. According to Crabb et al. (Crabb J W. et al., (1998), Protein Sci. 7(3): 746-57) the absorption maximum at 280 nm corresponds to the protein concentration, and the one at 400 nm to bound 9-cis retinal. The fully saturated CRALBP in complex with 9-cis retinal has been reported by the later to have an ideal spectral ratio of ε²⁸⁰/ε⁴⁰⁰=2.2 In our experiments, the ratios for wild-type CRALBP:9-cis retinal and for the A212C:T250C:9-cis retinal mutant are 2.31 and 2.21, respectively, indicating the formation of fully saturated 1:1 complexes (see FIG. 6).

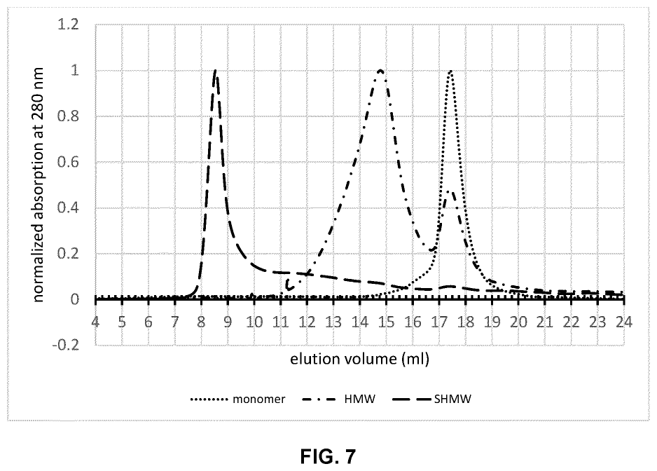

Analytical GFC of the pooled fractions under each peak were concentrated (approx. 4 mg/ml) and characterized with a Superose® 6 Increase 10/300 GL column. FIG. 7 shows overlayed traces at 280 nm of the SHMW, HMW and the monomeric analytical GFC run.

### Example 8

### Gel Filtration Chromatography of Di-Cys Mutants of CRALBP Under Oxidizing Conditions

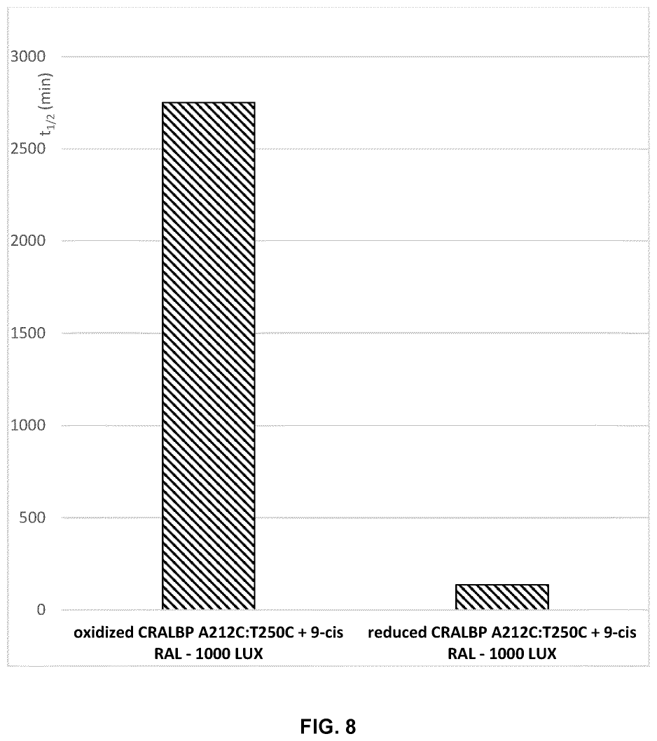

Purification of the protein cis-retinal complex of di-cysteine mutant A212C:T250C of CRALBP under oxidizing conditions was performed on a Superdex® 200 26/60 column in Gel filtration chromatography (GFC) buffer (10 mM Hepes, 100 mM NaCl, 5 mM GSSG, pH 7.5). The total elution volume was 330 ml, and fractions were collected at 5 ml intervals. Preparative gel filtration revealed essentially identical chromatograms when using the reduced cis-retinal complex of the di-cysteine mutant A212C: T250C of CRALBP or the cis-retinal complex of wild-type CRALBP (see Example 7, FIGS. 5 to 7). Oxidized glutathione (GSSG) is used during Gel filtration chromatography to in-situ generate oxidized di-cysteine CRALBP/cis-retinal complexes exhibiting about 20-fold increased photoprotection of the bound cis-retinal compared to the corresponding reduced complexes, see FIG. 8.

### Example 9

### Photoisomerization Assay

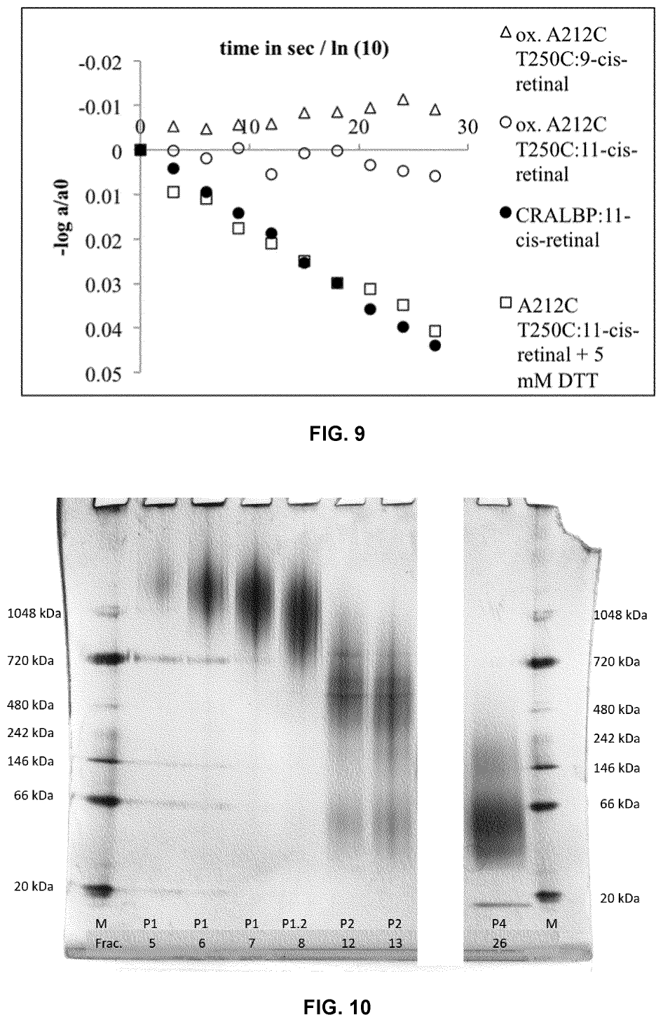

Photoisomerization assays of cis-retinal and its complexes with wild-type CRALBP or with di-cysteine mutant A212C:T250C of CRALBP were performed essentially as described by Saari et al. (Saari J C and Bredberg D L, (1978), J Biol Chem. 262(16):7618-22). For this, the gel permeation chromatography purified ligand complexes were diluted to 26 μM in (GFC) buffer (10 mM Hepes, 100 mM NaCl, pH 7.5) and equimolar amounts of BSA were added to avoid protein precipitation through the light induced formation of free all-trans-retinal. The samples were exposed with a 100-W daylight bulb to an illuminance of 380 lux (Voltcraft MS-1300 Luxmeter) at room temperature in the darkroom. Simultaneously UV/Vis absorption spectra were collected every 180 seconds for 3600 sec in total using the Evolution array UV/Vis spectrophotometer (Thermo Scientific). For photoisomerization experiments of the oxidized mutant A212C: T250C or the wild-type CRALBP, 5 mM oxidized glutathione was added to the preformed complexes and the mixtures were kept overnight at 4° C. For repeated reduction, the oxidized glutathion was first removed from the samples using a Vivaspin 15R Hydrosart (Sartorius) by three times concentrating the samples to a tenth of the volume and subsequent redilution in buffer (10 mM Hepes, 100 mM NaCl, pH 7.5) to the original volume. The washed samples were then reduced by adding 5 mM DTT and kept at 4° C. for 1 hour. As can be seen from FIG. 9, there was blockage of the mobile gate in the oxidized state, slowing down the rate of photoisomerization by at least 10-fold. Repeat reduction of the sample in 5 mM DTT re-established wild-type like behavior of the protein as captured by the assay. Thus, the introduction of the A212C:T250C double mutation represents an engineered redox-sensitive on-off switch into human CRALBP allowing for a reversible turning on and off of CRALBP's mobile gate functionality. The two mutations are located at adjacent positions in the mobile gate's interface of CRALBP allowing for the formation of an intramolecular disulfide bond under oxidizing conditions and to regain the gate's native functionality under reducing conditions. Accordingly, the photo-isomerization assay of the oxidized state of the A212C:T250C:11-cis-retinal complex reveals increased photoprotection for bound 11-cis-retinal, while the reduced state restores CRALBP's native in vitro photosensitivity.

### Example 10

### Native Page of Di-Cys Mutants of CRALBP

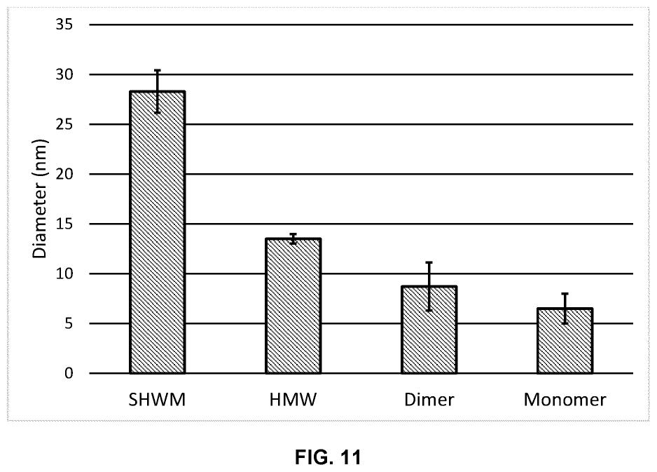

Native-PAGE of the di-cysteine mutants of CRALBP in complex with cis-retinal on a 4-16% polyacrylamide gel revealed similar bands as for wild-type CRALBP in complex with cis-retinal. After preparative GFC, the Native PAGE of the di-cysteine mutant A212C:T250C CRALPB samples showed the same protein band pattern for fractions derived from Peaks 1,2,3 and 4 as for wild-type CRALBP. The lanes containing fractions 5, 6, 7, and 8 from peak one and its right-sided shoulder (P1 and P1.2) revealed continuous protein bands starting at a molecular weight of 720 kDa and ranging beyond 1048 kDa, as shown in FIG. 10. These bands represent a A212C:T250C CRALBP oligomeric complex class, we call super-high molecular weight (SHMW). Lanes of the following smaller CRALBP oligomeric complex fraction are called high molecular weight CRALBP (HMW). These includes samples of the second peak (P2), fractions 12 and 13, and do feature a continuous band with a molecular weight between 242 kDa and 720 kDa, respectively.

### Example 11

### Characterization of Inventive Complexes by Dynamic Light Scattering

The same fractions derived from the preparative GFC that were characterized by analytical GFC, were also analyzed by dynamic light scattering (DLS) on a Malvern Zetasizer S®. Each DLS experiment contains was performed following the settings compiled in Table 5, the run duration and number of runs per measurement was set according to the auto-optimization of the Malvern Zetasizer software.

TABLE 5
Instruments settings for characterization by DLS

| | CRALBP A212C:T250C + |
|---|---|
| Application | 9-cis-retinal complexes |
| Type: | Protein Size |
| Material: | Protein |
| Refractive. Index RI: | 1.40 |
| Absorbance (Abs): | 0.001 |
| Dispersant: | H₂O |
| Temperature: | 25° C. |
| Viscosity: | 0.8872 cP |
| Refracting Index RI: | 1.33 |
| Equilibration time: | 15 sec |
| Detection angle: | 173° (backscatter) |
| Run duration: | Automatic |
| Number of run per measurement: | Automatic (10-15) |

The dynamic light scattering (DLS) measurements indicate that the sample peaks obtained from gel filtration represent discrete populations of inventive compositions and complexes, respectively. The average size distribution in diameter for the monomeric complex of di-cysteine A212C:T250C CRALBP with 9-cis-retinal is 6.50±1.50 nm, for the dimeric complex 8.72±2.41 nm, for the HMW 13.50±0.47 nm, and for the SHMW 28.28±2.13 nm, respectively (see FIG. 11).

1. A composition comprising, preferably consisting of, a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond; and
(b) a cognate ligand of CRALBP, wherein preferably said cognate ligand is a cis-retinoid.

2. The composition of claim 1, wherein one cysteine of each pair of amino acid mutations by cysteines is a mutation of an amino acid within the amino acid residues corresponding to amino acids 204-229 of SEQ ID NO:3, wherein the other mutated amino acid by cysteine of said pair is a mutation of an amino acid within the amino acid residues corresponding to amino acids 244-261 of SEQ ID NO:3.

3. The composition of claim 1 or claim 2, wherein said mutein comprises one, two, three or four pairs of amino acid mutations by cysteines as compared to said wild-type CRALBP protein, and wherein preferably said mutein comprises one or two pairs of amino acid mutations by cysteines as compared to said wild-type CRALBP protein.

4. The composition of any one of the preceding claims, wherein said pair of amino acid mutations by cysteines as compared to said wild-type CRALBP protein is selected from
(1) a mutation of an amino acid corresponding to amino acid 212 of SEQ ID NO:3 and a mutation of an amino acid corresponding to amino acid 250 of SEQ ID NO:3;
(2) a mutation of an amino acid corresponding to amino acid 217 of SEQ ID NO:3 and a mutation of an amino acid corresponding to amino acid 253 of SEQ ID NO:3;
(3) a mutation of an amino acid corresponding to amino acid 220 of SEQ ID NO:3 and a mutation of an amino acid corresponding to amino acid 254 of SEQ ID NO:3;
(4) a mutation of an amino acid corresponding to amino acid 224 of SEQ ID NO:3 and a mutation of an amino acid corresponding to amino acid 257 of SEQ ID NO:3.

5. The composition of any one of the claims 1 to 4, wherein said complex is a monomeric complex of said CRALBP mutant protein and said cognate ligand.

6. The composition of any one of the claims 1 to 4, wherein said complex is an oligomeric complex of said CRALBP mutant protein and said cognate ligand, wherein said oligomeric complex has a molecular weight of at least 600 kDa, preferably of at least 720 kDa, and preferably a molecular weight of at most 2500 kDa, further preferably a molecular weight of at most 2000 kDa, or wherein said oligomeric complex has a average diameter of about 24 to 33 nm, and wherein preferably said oligomeric complex has a average diameter of about 25 to 32 nm, wherein said average diameter is determined by Dynamic Light Scattering (DLS).

7. The composition of any one of the claims 1 to 4, wherein said complex comprises monomeric complexes and homo oligomeric complexes of said CRALBP mutant protein and said cognate ligand.

8. The composition of any one of the preceding claims, wherein each of said pair of cysteines forms a disulfide bond.

9. The composition of any one of the preceding claims, wherein said wild-type CRALBP protein is the human CRALBP protein of SEQ ID NO:3.

10. The composition of any one of the preceding claims, wherein said wild-type CRALBP protein is the human CRALBP protein of SEQ ID NO:3, and said mutein comprises one or two or three pairs of amino acid mutations by cysteines as compared to said human wild-type CRALBP protein, and wherein said one pair of amino acid mutations by cysteines as compared to said human wild-type CRALBP protein is selected from
(i) a mutation of amino acid 212 of SEQ ID NO:3 and a mutation of amino acid 250 of SEQ ID NO:3;
(ii) a mutation of amino acid 217 of SEQ ID NO:3 and a mutation of amino acid 253 of SEQ ID NO:3; and
(iii) a mutation of amino acid 220 of SEQ ID NO:3 and a mutation of amino acid 254 of SEQ ID NO:3;
and wherein said two pairs of amino acid mutations by cysteines as compared to said human wild-type CRALBP protein are selected from
(a) a first pair of amino acid mutations by cysteines and a second pair of amino acid mutations by cysteines, wherein said first pair of amino acid mutations by cysteines is a mutation of amino acid 220 of SEQ ID NO:3 and a mutation of amino acid 254 of SEQ ID NO:3, and said second pair of amino acid mutations by cysteines is a mutation of amino acid 224 of SEQ ID NO:3 and a mutation of amino acid 257 of SEQ ID NO:3;
(b) a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 224 of SEQ ID NO:3 and a mutation of amino acid 257 of SEQ ID NO:3; and
(c) a first pair of amino acid mutations by cysteines and a second pair of amino acid mutations by cysteines, wherein said first pair of amino acid mutations by cysteines is a mutation of amino acid 212 of SEQ ID NO:3 and a mutation of amino acid 250 of SEQ ID NO:3, and said second pair of amino acid mutations by cysteines is a mutation of amino acid 217 of SEQ ID NO:3 and a mutation of amino acid 253 of SEQ ID NO:3;
and wherein said three pairs of amino acid mutations by cysteines as compared to said human wild-type CRALBP protein is
(x) a first pair of amino acid mutations by cysteines, a second pair of amino acid mutations by cysteines, and a third pair of amino acid mutations by cysteines, wherein said first pair of amino acid mutations by cysteines is a mutation of amino acid 212 of SEQ ID NO:3 and a mutation of amino acid 250 of SEQ ID NO:3, said second pair of amino acid mutations by cysteines is a mutation of amino acid 220 of SEQ ID NO:3 and a mutation of amino acid 254 of SEQ ID NO:3, and said third pair of amino acid mutations by cysteines is a mutation of amino acid 224 of SEQ ID NO:3 and a mutation of amino acid 257 of SEQ ID NO:3.

11. The composition of any one of the preceding claims, wherein said CRALBP mutant protein has an amino acid sequence selected from group consisting of SEQ ID NO:9, SEQ ID NO:11, SEQ ID NO: 13, SEQ ID NO:15, SEQ ID NO: 17, SEQ ID NO: 19, SEQ ID NO:21 and SEQ ID NO:23, and wherein preferably said CRALBP mutant protein has an amino acid sequence selected from SEQ ID NO:9 and SEQ ID NO:21, and wherein further preferably said CRALBP mutant protein has the amino acid sequence of SEQ ID NO:9.

12. A CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond.

13. A nucleic acid sequence encoding for a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond.

14. A method of preparing a composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP;
wherein said method comprises the steps of
i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
iv. allowing said CRALBP mutant protein and said cognate ligand of CRALBP to assemble into a complex;
V. separating said composition comprising said complex from said solution III;
vi. optionally purifying said composition comprising said complex.

15. A composition comprising a complex, wherein said complex comprises
(a) a CRALBP mutant protein, wherein said CRALBP mutant protein is a mutein of a wild-type CRALBP protein, wherein said mutein comprises at least one pair of amino acid mutations by cysteines as compared to said wild-type CRALBP protein, wherein each of said pair of cysteines is able of forming a disulfide bond;
(b) a cognate ligand of CRALBP, wherein preferably said cognate ligand is a cis-retinoid;
wherein said composition is preferably defined as in any one of the claims 2 to 11; and wherein said composition is obtained by a method comprises the steps of
i. providing said CRALBP mutant protein in an aqueous solution I, wherein the concentration of said CRALBP mutant protein in said solution I is 1 μM to 5 mM, and wherein the pH of said solution I is 5 to 9, preferably 7.0-8.5, further preferably 7.5-8.5, and wherein preferably said solution I comprises a salt, wherein the concentration of said salt is 10 mM to 500 mM;
ii. providing said cognate ligand of CRALBP in a solution II, wherein the concentration of said cognate ligand of SEC14-like protein in said solution I is 5 μM to 500 mM, and wherein the solvent of said solution II is a water soluble solvent, wherein preferably said water soluble solvent is ethanol;
iii. generating a solution III by combining said solution I and said solution II, wherein the ratio of the concentration of said CRALBP mutant protein and the concentration of said cognate ligand of CRALBP in said solution III is of between 4:1 to 1:4 (molar/molar), and wherein the volume of said water soluble solvent in said solution III is of between 0.5-8% (vol/vol), preferably of between about 1-5% (vol/vol);
iv. allowing said CRALBP mutant protein and said cognate ligand of CRALBP to assemble into a complex;
v. separating said composition comprising said complex from said solution III;
vi. optionally purifying said composition comprising said complex.
